(19) United States
(12) Patent Application Publication
(10) Pub. No.: US 2022/0142892 A1
(43) Pub. Date: May 12, 2022

(54) PROCESS FOR THE PREPARATION AND USE OF HAIR TREATMENT COMPOSITIONS CONTAINING ORGANIC C1-C6 ALKOXY SILANES

(71) Applicant: Henkel AG & Co. KGaA, Duesseldorf (DE)

(72) Inventors: Torsten LECHNER, Langenfeld (DE); Juergen SCHOEPGENS, Schwalmtal (DE)

(73) Assignee: Henkel AG & Co. KGaA, Duesseldorf (DE)

(21) Appl. No.: 17/436,557

(22) PCT Filed: Jan. 8, 2020
(86) PCT No.: PCT/EP2020/050314

§ 371 Date: Sep. 3, 2021

(30) Foreign Application Priority Data
Mar. 6, 2019 (DE) 102019203079.3

(51) Int. Cl. A61K 8/58; A61K 8/34; A61Q 5/06; A61Q 5/10

(52) U.S. Cl. CPC A61K 8/585; A61K 8/34; A61Q 5/065; A61K 2800/4324; A61K 2800/805; A61K 2800/882; A61K 2800/4322; A61Q 5/10

(57) ABSTRACT

The subject of the present application is a process for the preparation of an agent for the treatment of keratinous material, in particular human hair, comprising the following steps:

(1) mixing one or more organic C₁-C₆ alkoxy silanes with water,
(2) optionally adding one or more cosmetic ingredients to the mixture obtained in step (1),
(3) storage of the mixture for a period of at least 5 days,
(4) if necessary, addition of one or more cosmetic ingredients to the stored mixture,
(5) partial or complete removal from the mixture of the C₁-C₆ alcohols released in steps (1) to (4).

### CROSS-REFERENCE TO RELATED APPLICATION

This application is a U.S. National-Stage entry under 35 U.S.C. § 371 based on International Application No. PCT/EP2020/050314, filed Jan. 8, 2020, which was published under PCT Article 21(2) and which claims priority to German Application No. 102019203079.3, filed Mar. 6, 2019, which are all hereby incorporated in their entirety by reference.

### TECHNICAL FIELD

The present application is in the field of cosmetics and relates to a process for the preparation and use of hair treatment compositions.

### BACKGROUND

The change in shape and color of keratin fibers, especially hair, is an important area of modern cosmetics. To change the hair color, the expert knows various coloring systems depending on coloring requirements. Oxidation dyes are usually used for permanent, intensive dyeing's with good fastness properties and good grey coverage. Such dyes usually contain oxidation dye precursors, so-called developer components and coupler components, which form the actual dyes with one another under the influence of oxidizing agents, such as hydrogen peroxide. Oxidation dyes are characterized by very long-lasting dyeing results.

When direct dyes are used, ready-made dyes diffuse from the colorant into the hair fiber. Compared to oxidative hair dyeing, the dyeing's obtained with direct dyes have a shorter shelf life and quicker wash ability. Dyeing with direct dyes usually remain on the hair for a period of between 5 and 20 washes.

The use of color pigments is known for short-term color changes on the hair and/or skin. Color pigments are generally understood to be insoluble, coloring substances. These are present undissolved in the dye formulation in the form of small particles and are only deposited from the outside on the hair fibers and/or the skin surface. Therefore, they can usually be removed again without residue by a few washes with detergents containing surfactants. Various products of this type are available on the market under the name hair mascara.

If the user wants particularly long-lasting dyeing's, the use of oxidative dyes has so far been his only option. However, despite numerous optimization attempts, an unpleasant ammonia or amine odor cannot be completely avoided in oxidative hair dyeing. The hair damage still associated with the use of oxidative dyes also has a negative effect on the user's hair.

EP 2168633 B1 deals with the task of producing long-lasting hair colorations using pigments. The paper teaches that when a combination of pigment, organic silicon compound, hydrophobic polymer and a solvent is used on hair, it is possible to produce colorations that are particularly resistant to shampooing.

The organic silicon compounds used in EP 2168633 B1 are reactive compounds from the class of alkoxy silanes. These alkoxy silanes hydrolyze at high rates in the presence of water and form hydrolysis products and/or condensation products, depending on the amounts of alkoxy silane and water used in each case. The influence of the amount of water used in this reaction on the properties of the hydrolysis or condensation product are described, for example, in WO 2013068979 A2.

When these hydrolysis or condensation products are applied to keratinous material, a film or coating is formed on the keratinous material, which completely envelops the keratinous material and, in this way, strongly influences the properties of the keratinous material. Possible areas of application include permanent styling or permanent shape modification of keratin fibers. In this process, the keratin fibers are mechanically shaped into the desired form and then fixed in this form by forming the coating described above. Another particularly suitable application is the coloring of keratin material; in this application, the coating or film is produced in the presence of a coloring compound, for example a pigment. The film colored by the pigment remains on the keratin material or keratin fibers and results in surprisingly wash-resistant colorations.

The great advantage of the alkoxy silane-based dyeing principle is that the high reactivity of this class of compounds enables extremely fast coating. This means that extremely good coloring results can be achieved after noticeably short application periods of just a few minutes. In addition to these advantages, however, the high reactivity of alkoxy silanes also has some disadvantages. Thus, even minor changes in production and application conditions, such as changes in humidity and/or temperature, can lead to sharp fluctuations in product performance. Most importantly, the work leading to this invention has shown that the alkoxy silanes are extremely sensitive to the conditions encountered during the manufacture and storage of the keratin treatment compositions.

If these manufacturing conditions deviate only slightly from their optimal range of values, this can lead to partial or even complete loss of product performance. In this context, it has also been found that the conditions prevailing during storage can also have a strong influence on the dyeing performance of an alkoxy silane-containing colorant.

### BRIEF SUMMARY

A process for the preparation of an agent for the treatment of keratinous material is provided. The process comprises a step of (1) mixing one or more organic C₁-C₆ alkoxy silanes with water to give a mixture. The process optionally comprises a step of (2) adding one or more cosmetic ingredients to the mixture. The process further comprises a step of (3) storing the mixture, from step (1), optionally from step (2), for a period of at least about 5 days to give a stored mixture. The process also optionally comprises a step of (4) adding one or more cosmetic ingredients to the stored mixture. The process further comprises a step of (5) partially or completely removing from the stored mixture one or more C₁-C₆ alcohols released by a reaction of the one or more organic C₁-C₆ alkoxy silanes and water mixed in step (1).

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

In the process as contemplated herein, one or more organic C₁-C₆ alkoxy silanes are reacted with water, and optionally one or more cosmetic ingredients are then added to the mixture. After that, the mixture is stored for a certain period, and a cosmetic ingredient can be added again. Subsequently, the C₁-C₆ alcohols released in the previous steps are partially or completely removed from the mixture.

It was the task of the present application to find an optimized process for the production and storage of keratin treatment agents. The alkoxy silanes used in this process were to be produced and stored in a targeted manner so that the optimum application properties could be achieved in a subsequent application. In particular, the agents prepared by this method should have improved dyeing performance, i.e., when used in a dyeing process, dyeing's with higher color intensity and improved fastness properties, especially improved wash fastness and improved rub fastness, should be obtained.

Surprisingly, it has now been found that the task can be excellently solved if the targeted hydrolysis is carried out by mixing the alkoxy silanes with water, then optionally adding a further cosmetic ingredient to this mixture and then storing the mixture under special conditions. Following storage of the mixture, a cosmetic ingredient can optionally be added again, followed by removal of the previously released alcohols from the mixture.

A first object of the present disclosure is a method for preparing an agent for treating keratinous material, in particular human hair, comprising the following steps:
(1) mixing one or more organic C₁-C₆ alkoxy silanes with water,
(2) if necessary, addition of one or more cosmetic ingredients to the mixture,
(3) storage of the mixture for a period of at least 5 days,
(4) where appropriate, addition of one or more cosmetic ingredients to the stored mixture; and
(5) partial or complete removal from the mixture of the C₁-C₆ alcohols released in steps (1) to (4).

It has been shown that hair treatment compositions prepared by this process as contemplated herein, when used in a dyeing process, resulted in very intense and uniform colorations with particularly good coverage, rub fastness and wash fastness.

A second object of the present disclosure is a multi-component packaging unit (kit-of-parts) for coloring keratinous material, which comprises, separately packaged in two packaging units, the cosmetic preparations (A) and (B), the preparation (A) being an agent of the first object of the invention and the preparation (B) containing at least one coloring compound.

### Agent for the Treatment of Keratinous Material

Keratinous material includes hair, skin, nails (such as fingernails and/or toenails). Wool, furs, and feathers also fall under the definition of keratinous material.

Preferably, keratinous material is understood to be human hair, human skin, and human nails, especially fingernails and toenails. Keratinous material is understood to be human hair.

Agents for treating keratinous material are understood to mean, for example, means for coloring the keratinous material, means for reshaping or shaping keratinous material, in particular keratinous fibers, or also means for conditioning or caring for the keratinous material. The agents prepared by the process as contemplated herein are particularly suitable for coloring keratinous material, in particular keratinous fibers, which are preferably human hair.

The term “coloring agent” is used in the context of the present disclosure to refer to a coloring of the keratin material, of the hair, caused using coloring compounds, such as thermochromic and photochromic dyes, pigments, mica, direct dyes and/or oxidation dyes. In this staining process, the colorant compounds are deposited in a particularly homogeneous and smooth film on the surface of the keratin material or diffuse into the keratin fiber. The film forms in situ by oligomerization or polymerization of the organic silicon compound(s), and by the interaction of the color-imparting compound and organic silicon compound and optionally other ingredients, such as a film-forming hydrophilic polymer.

Mixing C₁-C₆ Alkoxy Silane(s) with Water

Step (1) of the process as contemplated herein involves the reaction or also reaction of one or more organic C₁-C₆ alkoxy silanes with water. To initiate this reaction, the C₁-C₆ alkoxy silane(s) are mixed with water.

The organic C₁-C₆ alkoxy silane(s) are organic, non-polymeric silicon compounds, preferably selected from the group of silanes containing one, two or three silicon atoms.

Organic silicon compounds, alternatively called organosilicon compounds, are compounds which either have a direct silicon-carbon bond (Si—C) or in which the carbon is bonded to the silicon atom via an oxygen, nitrogen, or sulfur atom. The organic silicon compounds as contemplated herein are preferably compounds containing one to three silicon atoms. Organic silicon compounds preferably contain one or two silicon atoms.

According to IUPAC rules, the term silane chemical compounds based on a silicon skeleton and hydrogen. In organic silanes, the hydrogen atoms are completely or partially replaced by organic groups such as (substituted) alkyl groups and/or alkoxy groups.

A characteristic feature of the C₁-C₆ alkoxy silanes as contemplated herein is that at least one C₁-C₆ alkoxy group is directly bonded to a silicon atom. The C₁-C₆ alkoxy silanes as contemplated herein thus comprise at least one structural unit R′R″R′″Si—O—(C₁-C₆ alkyl) where the radicals R′, R″ and R′″ stand for the three-remaining bond valencies of the silicon atom.

The C₁-C₆ alkoxy group or groups bonded to the silicon atom are very reactive and are hydrolyzed at high rates in the presence of water, the reaction rate depending, among other things, on the number of hydrolyzable groups per molecule. If the hydrolyzable C₁-C₆ alkoxy group is an ethoxy group, the organic silicon compound preferably contains a structural unit R′R″R′″Si—O—CH2-CH3. The R′, R″ and R′″ residues again represent the three remaining free valences of the silicon atom.

In a very particularly preferred embodiment, a process as contemplated herein is exemplified in that in step (1), one or more organic C₁-C₆ alkoxy silanes selected from silanes having one, two or three silicon atoms are reacted with water, the organic silicon compound further comprising one or more basic chemical functions.

This basic group can be, for example, an amino group, an alkylamino group or a dialkylamino group, which is preferably connected to a silicon atom via a linker. Preferably, the basic group is an amino group, a C₁-C₆ alkylamino group or a di(C₁-C₆)alkylamino group.

A very particularly preferred method as contemplated herein exemplified by the (1) mixing one or more organic C₁-C₆ alkoxy silanes with water, wherein the organic C₁-C₆ alkoxy silanes are selected from the group of silanes having one, two or three silicon atoms, and wherein the C₁-C₆ alkoxy silanes further comprise one or more basic chemical functions.

Particularly good results were obtained when C₁-C₆ alkoxy silanes of formula (I) and/or (II) were used in the process as contemplated herein.

In another very particularly preferred embodiment, a method as contemplated herein is exemplified by the

(1) mixing one or more organic C₁-C₆ alkoxy silanes of formula (I) and/or (II) with water,

R₁R₂N-L-Si(OR₃)ₐ(R₄)_{b} (I)

where
R₁, R₂ independently represent a hydrogen atom or a C₁-C₆ alkyl group,
L is a linear or branched divalent C₁-C₂₀ alkylene group,
R₃, R₄ independently of one another represent a C₁-C₆ alkyl group,
a, stands for an integer from 1 to 3, and
b stands for the integer 3-a, and

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),

where
R5, R5′, R5″, R6, R6′ and R6″ independently represent a C₁-C₆ alkyl group,
A, A′, A″, A′″ and A″″ independently represent a linear or branched divalent C₁-C₂₀ alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III),

(A″″)-Si(R₆″)_{d}“(OR₅″)_{c}” (III),
c, stands for an integer from 1 to 3,
d stands for the integer 3-c,
c′ stands for an integer from 1 to 3,
d′ stands for the integer 3-c′,
c″ stands for an integer from 1 to 3,
d″ stands for the integer 3-c″,
e stands for 0 or 1,
f stands for 0 or 1,
g stands for 0 or 1,
h stands for 0 or 1,
provided that at least one of e, f, g, and h is different from 0.

The substituents R₁, R₂, R₃, R₄, R₅, R₅′, R₅″, R₆, R₆′, R₆″, R₇, R₈, L, A, A′, A″. A′″ and A″″ in the compounds of formula (I) and (II) are explained below as examples:

Examples of a C₁-C₆ alkyl group are the groups methyl, ethyl, propyl, isopropyl, n-butyl, s-butyl, and t-butyl, n-pentyl and n-hexyl. Propyl, ethyl, and methyl are preferred alkyl radicals. Examples of a C₂-C₆ alkenyl group are vinyl, allyl, but-2-enyl, but-3-enyl and isobutenyl, preferred C₂-C₆ alkenyl radicals are vinyl and allyl. Preferred examples of a hydroxy C₁-C₆ alkyl group are a hydroxymethyl, a 2-hydroxyethyl, a 2-hydroxypropyl, a 3-hydroxypropyl, a 4-hydroxybutyl group, a 5-hydroxypentyl and a 6-hydroxyhexyl group; a 2-hydroxyethyl group is particularly preferred. Examples of an amino C₁-C₆ alkyl group are the aminomethyl group, the 2-aminoethyl group, the 3-aminopropyl group. The 2-aminoethyl group is particularly preferred. Examples of a linear divalent C₁-C₂₀ alkylene group include the methylene group (—CH₂)), the ethylene group (—CH₂—CH₂—), the propylene group (—CH₂—CH₂—CH₂—) and the butylene group (—CH₂—CH₂—CH₂—CH₂—). The propylene group (—CH₂—CH₂—CH₂—) is particularly preferred. From a chain length of 3 C atoms, divalent alkylene groups can also be branched. Examples of branched divalent C₃-C₂₀ alkylene groups are (—CH₂—CH(CH₃)—) and (—CH₂—CH(CH₃)—CH₂—).

In the organic silicon compounds of the formula (I)

R₁R₂N-L-Si(OR₃)ₐ(R₄)_{b} (I),

the radicals R₁ and R₂ independently of one another represent a hydrogen atom or a C₁-C₆ alkyl group. In particular, the radicals R₁ and R₂ both represent a hydrogen atom.

In the middle part of the organic silicon compound is the structural unit or the linker -L- which stands for a linear or branched, divalent C₁-C₂₀ alkylene group. The divalent C₁-C₂₀ alkylene group may alternatively be referred to as a divalent or divalent C₁-C₂₀ alkylene group, by which is meant that each—L grouping may form—two bonds.

Preferably -L- stands for a linear, divalent C₁-C₂₀ alkylene group. Further preferably -L- stands for a linear divalent C₁-C₆ alkylene group. Particularly preferred -L stands for a methylene group (CH₂—), an ethylene group (—CH₂—CH₂—), propylene group (—CH₂—CH₂—CH₂—) or butylene (—CH₂—CH₂—CH₂—CH₂—). L stands for a propylene group (—CH₂—CH₂—CH₂—)

The organic silicon compounds of formula (I)

R₁R₂N-L-Si(OR₃)ₐ(R₄)_{b} (I),

one end of each carries the silicon-containing group —Si(OR₃)ₐ(R₄)_{b}.

In the terminal structural unit —Si(OR₃)ₐ(R₄)_{b}, R₃ and R₄ independently represent a C₁-C₆ alkyl group, and particularly preferably R₃ and R₄ independently represent a methyl group or an ethyl group.

Here a stands for an integer from 1 to 3, and b stands for the integer 3-a. If a stands for the number 3, then b is equal to 0. If a stands for the number 2, then b is equal to 1. If a stands for the number 1, then b is equal to 2.

Keratin treatment agents with particularly good properties could be prepared if in step (1) at least one organic C₁-C₆ alkoxy silane of formula (I) was mixed with water or reacted, in which the radicals R₃, R₄ independently of one another represent a methyl group or an ethyl group.

Furthermore, dyeings with the best wash fastnesses could be obtained when at least one organic C₁-C₆ alkoxy silane of formula (I) was reacted with water in step (1), in which the radical a represents the number 3. In this case the rest b stands for the number 0.

In another preferred embodiment, a process as contemplated herein is exemplified in that in step (1) one or more organic C₁-C₆ alkoxy silanes of formula (I) are mixed with water,

where

R₃, R₄ independently of one another represent a methyl group or an ethyl group and

a stands for the number 3 and

b stands for the number 0.

In a further preferred embodiment, a process as contemplated herein is exemplified in that in step (1) one or more organic C₁-C₆ alkoxy silanes of formula (I) and/or (II) are mixed or reacted with water,

R₁R₂N-L-Si(OR₃)ₐ(R₄)_{b} (I),

where
R₁, R₂ both represent a hydrogen atom, and
L represents a linear, divalent C₁-C₆-alkylene group, preferably a propylene group (—CH₂—CH₂—CH₂—) or an ethylene group (—CH₂—CH₂—),
R₃ represents an ethyl group or a methyl group,
R₄ represents a methyl group or an ethyl group,
a stands for the number 3 and
b stands for the number 0.

Organic silicon compounds of the formula (I) which are particularly suitable for solving the problem as contemplated herein are

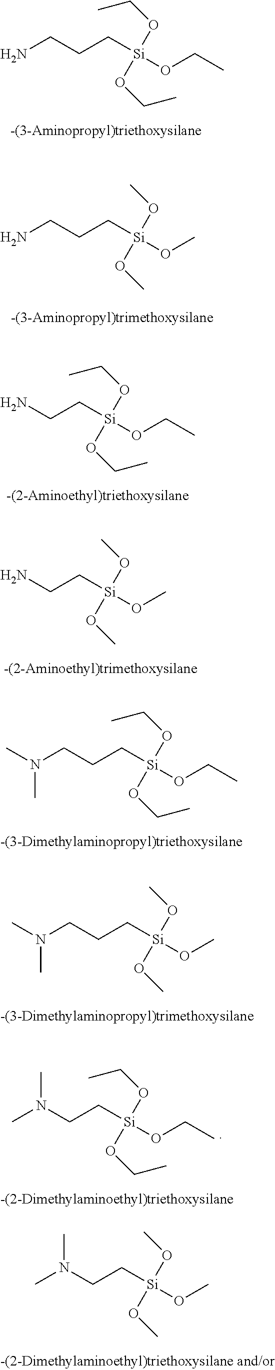

In a further preferred embodiment, a process as contemplated herein is exemplified in that in step (1) one or more organic C₁-C₆ alkoxy silanes is selected from the group including
(3-Aminopropyl)triethoxysilane
(3-Aminopropyl)trimethoxysilane
(2-Aminoethyl)triethoxysilane
(2-Aminoethyl)trimethoxysilane
(3-Dimethylaminopropyl)triethoxysilane
(3-Dimethylaminopropyl)trimethoxysilane
(2-Dimethylaminoethyl)triethoxysilan.
(2-Dimethylaminoethyl)trimethoxysilane and/or
   mixed with water or made to react.

The organic silicon compound of formula (I) is commercially available. (3-aminopropyl)trimethoxysilane, for example, can be purchased from Sigma-Aldrich. Also (3-aminopropyl)triethoxysilane is commercially available from Sigma-Aldrich.

In a further embodiment of the process as contemplated herein, one or more organic C₁-C₆ alkoxy silanes of formula (II) may also be mixed with water or reacted in step (1),

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II).

The organosilicon compounds of formula (II) as contemplated herein each carry the silicon-containing groups (R₅O)_{c}(R₆)_{d}Si— and —Si(R₆′)_{d′}(OR₅′)_{c} at both ends.

In the central part of the molecule of formula (II) there are the groups -(A)ₑ- and —[NR₇-(A′)]_{f}- and —[O-(A″)]_{g}- and —[NR₈-(A′″)]ₕ-. Here, each of the radicals e, f, g, and h can independently of one another stand for the number 0 or 1, with the proviso that at least one of the radicals e, f, g, and h is different from 0. In other words, an organic silicon compound of formula (II) as contemplated herein contains at least one grouping from the group including -(A)- and —[NR₇-(A′)]- and —[O-(A″)]- and —[NR₈-(A′″)]-.

In the two terminal structural units (R₅O)_{c}(R₆)_{d}Si— and —Si(R₆′)_{d′}(OR₅′)_{c}′, the residues R5, R5′, R5″ independently represent a C₁-C₆ alkyl group. The radicals R6, R6′ and R6″ independently represent a C₁-C₆ alkyl group.

Here, a stands for an integer from 1 to 3, and d stands for the integer 3-c. If c stands for the number 3, then d is equal to 0. If c stands for the number 2, then d is equal to 1. If c stands for the number 1, then d is equal to 2.

Analogously c′ stands for a whole number from 1 to 3, and d′ stands for the whole number 3-c′. If c′ stands for the number 3, then d′ is 0. If c′ stands for the number 2, then d′ is 1. If c′ stands for the number 1, then d′ is 2.

Dyeings with the best wash fastness values could be obtained if the residues c and c′ both stand for the number 3. In this case d and d′ both stand for the number 0.

In a further preferred embodiment, a process as contemplated herein is exemplified in that in step (1) one or more organic C₁-C₆ alkoxy silanes of formula (II) are mixed or reacted with water,

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),

where
R5 and R5′ independently represent a methyl group or an ethyl group,
c and c′ both stand for the number 3 and
d and d′ both stand for the number 0.

If c and c′ are both the number 3 and d and d′ are both the number 0, the organic silicon compound as contemplated herein corresponds to formula (IIa)

(R₅O)₃Si-(A)ₑ-[NR₇-(A′)]_{f}-[O-(A″)]_{g}-[NR₈-(A′″)]ₕ—Si(OR₅′)₃ (IIa).

The radicals e, f, g, and h can independently stand for the number 0 or 1, whereby at least one radical from e, f, g, and h is different from zero. The abbreviations e, f, g, and h thus define which of the groupings -(A)ₑ- and -[NR7-(A′)]_{f}- and —[O-(A″)]_{g}- and -[NR8-(A′″)]ₕ- are in the middle part of the organic silicon compound of formula (II).

In this context, the presence of certain groupings has proven to be particularly advantageous in terms of achieving washfast dyeing results. Particularly good results could be obtained if at least two of the residues e, f, g, and h stand for the number 1. Especially preferred e and f both stand for the number 1. Furthermore, g and h both stand for the number 0.

If e and f both stand for the number 1 and g and h both stand for the number 0, the organic silicon compound as contemplated herein corresponds to formula (IIb)

(R₅O)_{c}(R₆)_{d}Si-(A)-[NR₇-(A′)]-Si(R₆′)_{d′}(OR₅′)_{c′} (IIb).

The radicals A, A′, A″, A′″ and A′″ independently represent a linear or branched divalent C₁-C₂₀ alkylene group. Preferably the radicals A, A′, A″, A′″ and A″″ independently of one another represent a linear, divalent C₁-C₂₀ alkylene group. Further preferably the radicals A, A′, A″, A′″ and A″″ independently represent a linear divalent C₁-C₆ alkylene group.

The divalent C₁-C₂₀ alkylene group may alternatively be referred to as a divalent or divalent C₁-C₂₀ alkylene group, by which is meant that each grouping A, A′, A″, A′″ and A″″ may form two bonds.

In particular, the radicals A, A′, A″, A′″ and A″″ independently of one another represent a methylene group (—CH₂—), an ethylene group (—CH₂—CH₂—), a propylene group (—CH₂—CH₂—CH₂—) or a butylene group (—CH₂—CH₂—CH₂—CH₂—). In particular, the radicals A, A′, A″, A′″ and A″″ stand for a propylene group (—CH₂—CH₂—CH₂—).

If the radical f represents the number 1, then the organic silicon compound of formula (II) as contemplated herein contains a structural grouping —[NR₇-(A′)]-.

If the radical f represents the number 1, then the organic silicon compound of formula (II) as contemplated herein contains a structural grouping —[NR₈-(A′″)]-.

Wherein R₇ and R₈ independently represent a hydrogen atom, a C₁-C₆ alkyl group, a hydroxy-C₁-C₆ alkyl group, a C₂-C₆ alkenyl group, an amino-C₁-C₆ alkyl group or a group of the formula (III)

-(A″″)-Si(R₆″)_{d′}(OR₅″)_{c}″ (III).

Very preferably the radicals R7 and R8 independently of one another represent a hydrogen atom, a methyl group, a 2-hydroxyethyl group, a 2-alkenyl group, a 2-aminoethyl group or a grouping of the formula (III).

If the radical f represents the number 1 and the radical h represents the number 0, the organic silicon compound as contemplated herein contains the grouping [NR₇-(A′)] but not the grouping —[NR₈-(A′″)]. If the radical R7 now stands for a grouping of the formula (III), the organic silicone compound comprises 3 reactive silane groups.

In a further preferred embodiment, a process as contemplated herein is exemplified in that in step (1) one or more organic C₁-C₆ alkoxy silanes of the formula (II) are reacted with water

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),

where
e and f both stand for the number 1,
g and h both stand for the number 0,
A and A′ independently represent a linear, divalent C₁-C₆ alkylene group and
R₇ represents a hydrogen atom, a methyl group, a 2-hydroxyethyl group, a 2-alkenyl group, a 2-aminoethyl group or a group of formula (III).

In a further preferred embodiment, a process as contemplated herein is exemplified in that in step (1) one or more organic C₁-C₆ alkoxy silanes of formula (II) are mixed or reacted with water, where

e and f both stand for the number 1,
g and h both stand for the number 0,
A and A′ independently of one another represent a methylene group (—CH₂—), an ethylene group (—CH₂—CH₂—) or a propylene group (—CH₂—CH₂—CH₂), and
R₇ represents a hydrogen atom, a methyl group, a 2-hydroxyethyl group, a 2-alkenyl group, a 2-aminoethyl group or a group of formula (III).

Organic silicon compounds of the formula (II) which are well suited for solving the problem as contemplated herein are

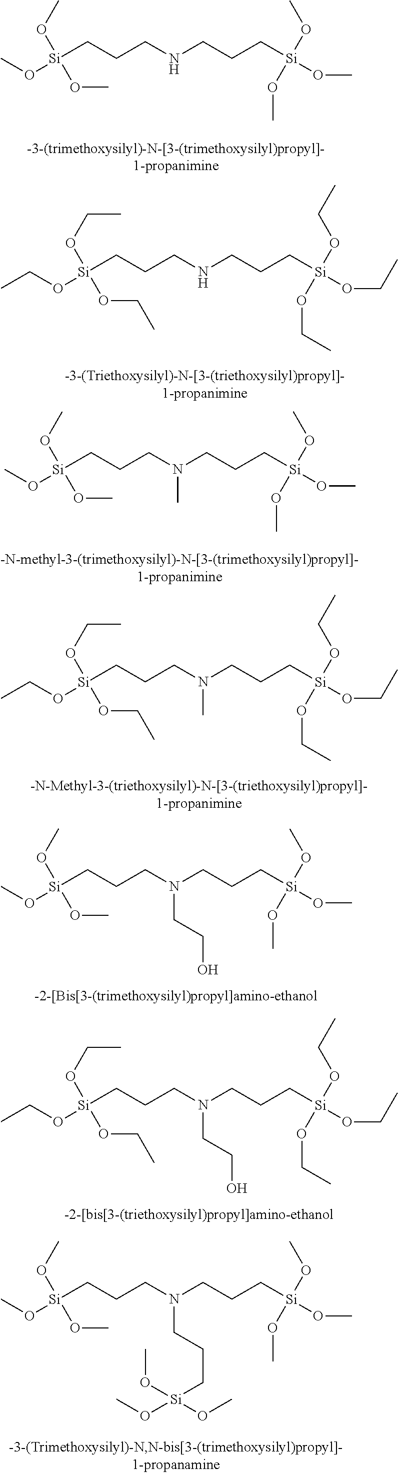

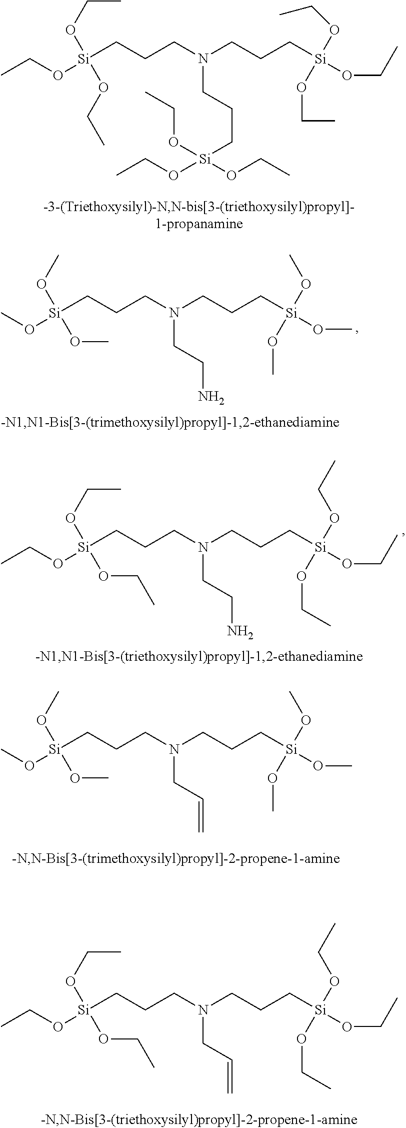

The organic silicon compounds of formula (II) are commercially available.

Bis(trimethoxysilylpropyl)amines with the CAS number 82985-35-1 can be purchased from Sigma-Aldrich.
Bis[3-(triethoxysilyl)propyl]amines with the CAS number 13497-18-2 can be purchased from Sigma-Aldrich, for example.
N-methyl-3-(trimethoxysilyl)-N-[3-(trimethoxysilyl)propyl]-1-propanamine is alternatively referred to as bis(3-trimethoxysilylpropyl)-N-methylamine and can be purchased commercially from Sigma-Aldrich or Fluorochem.
3-(triethoxysilyl)-N,N-bis[3-(triethoxysilyl)propyl]-1-propanamine with the CAS number 18784-74-2 can be purchased for example from Fluorochem or Sigma-Aldrich.

In a further preferred embodiment, a process as contemplated herein is exemplified in that in step (1) one or more organic C₁-C₆ alkoxy silanes of formula (II) selected from the group including
3-(trimethoxysilyl)-N-[3-(trimethoxysilyl) propyl]-1-propanamine
3-(Triethoxysilyl)-N-[3-(triethoxysilyl) propyl]-1-propanamine
N-methyl-3-(trimethoxysilyl)-N-[3-(trimethoxysilyl) propyl]-1-propanamine
N-Methyl-3-(triethoxysilyl)-N-[3-(triethoxysilyl) propyl]-1-propanamine
2-[Bis[3-(trimethoxysilyl) propyl]amino]-ethanol
2-[bis[3-(triethoxysilyl) propyl]amino]ethanol
3-(Trimethoxysilyl)-N,N-bis[3-(trimethoxysilyl) propyl]-1-propanamine
3-(Triethoxysilyl)-N,N-bis[3-(triethoxysilyl) propyl]-1-propanamine
N1,N1-bis[3-(trimethoxysilyl) propyl]-1,2-ethanediamine,
N1,N1-bis[3-(triethoxysilyl) propyl]-1,2-ethanediamine,
N,N-bis[3-(trimethoxysilyl)propyl]-2-propen-1-amine and/or
N,N-bis[3-(triethoxysilyl)propyl]-2-propene-1-,
   be reacted with water or mixed with water.

In further dyeing trials, it has also been found to be particularly advantageous if at least one organic C₁-C₆ alkoxy silane of the formula (IV) was used in the process as contemplated herein

R₉Si(OR₁₀)ₖ(R₁₁)ₘ (IV).

The compounds of formula (IV) are organic silicon compounds selected from silanes having one, two or three silicon atoms, wherein the organic silicon compound comprises one or more hydrolyzable groups per molecule.

The organic silicon compound(s) of formula (IV) may also be referred to as silanes of the alkyl-C₁-C₆-alkoxy-silane type,

R₉Si(OR₁₀)ₖ(R₁₁)ₘ (IV),

where
R₉ represents a C₁-C₁₂ alkyl group,
R₁₀ represents a C₁-C₆ alkyl group,
R₁₁ represents a C₁-C₆ alkyl group
k is an integer from 1 to 3, and
m stands for the integer 3-k.

In a further embodiment, a particularly preferred method as contemplated herein is exemplified by the

(1) mixing one or more organic C₁-C₆ alkoxy silanes of formula (IV) with water,

R₉Si(OR₁₀)ₖ(R₁₁)ₘ (IV),

where
R₉ represents a C₁-C₁₂ alkyl group,
R₁₀ represents a C₁-C₆ alkyl group,
R₁₁ represents a C₁-C₆ alkyl group
k is an integer from 1 to 3, and
m stands for the integer 3-k.

In the organic C₁-C₆ alkoxy silanes of formula (IV), the R₉ radical represents a C₁-C₁₂ alkyl group. This C₁-C₁₂ alkyl group is saturated and can be linear or branched. Preferably R₉ stands for a linear C₁-C₈ alkyl group. Preferably R₉ stands for a methyl group, an ethyl group, an n-propyl group, an n-butyl group, an n-pentyl group, an n-hexyl group, an n-octyl group or an n-dodecyl group. Particularly preferred, R₉ stands for a methyl group, an ethyl group or an n-octyl group.

In the organic silicon compounds of formula (IV), the radical R₁₀ represents a C₁-C₆ alkyl group. R₁₀ stands for a methyl group or an ethyl group.

In the organic silicon compounds of formula (IV), the radical R₁₁ represents a C₁-C₆ alkyl group. R₁₁ stands for a methyl group or an ethyl group.

Furthermore, k stands for a whole number from 1 to 3, and m stands for the whole number 3-k. If k stands for the number 3, then m is equal to 0. If k stands for the number 2, then m is equal to 1. If k stands for the number 1, then m is equal to 2.

Dyeings with the best wash fastnesses could be obtained if at least one organic silicon compound of formula (IV), in which the radical k represents the number 3, was used in the preparation of the preparation as contemplated herein. In this case the rest m stands for the number 0.

Organic silicon compounds of the formula (IV) which are particularly suitable for solving the problem as contemplated herein are

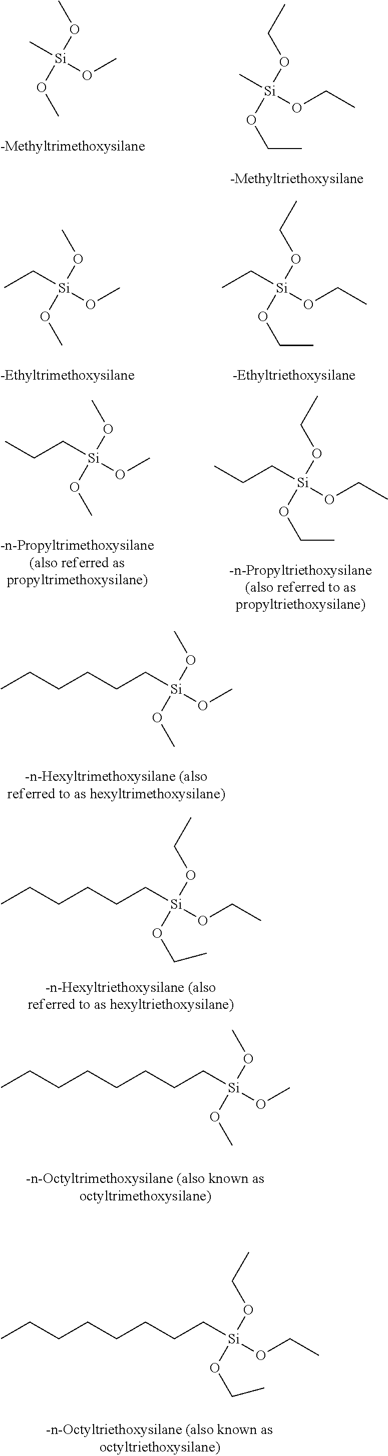

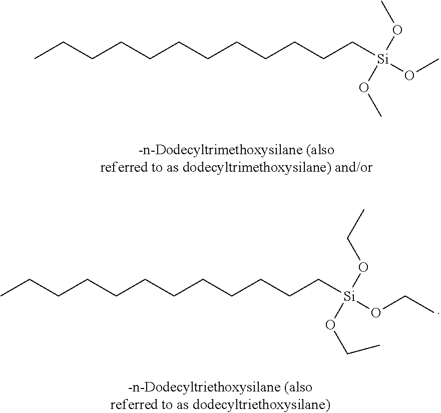

In a further preferred embodiment, a process as contemplated herein is exemplified in that in step (1) one or more organic C₁-C₆ alkoxy silanes of formula (IV) selected from the group including
Methyltrimethoxysilane
Methyltriethoxysilane
Ethyltrimethoxysilane
Ethyltriethoxysilane
Propyltrimethoxysilane
Propyltriethoxysilane
Hexyltrimethoxysilane
Hexyltriethoxysilane
Octyltrimethoxysilane
Octyltriethoxysilane
Dodecyltrimethoxysilane and/or
Dodecyltriethoxysilane,
   mixed with water or reacted with water.

The process as contemplated herein can be carried out in a reaction vessel or reactor suitable for this purpose. Depending on the desired approach size, various prior art models are known and commercially available for this purpose.

For example, the reaction of the organic C₁-C₆ alkoxy silanes with water can be carried out in a reaction vessel or a reactor, preferably a double-walled reactor, a reactor with an external heat exchanger, a tubular reactor, a reactor with a thin-film evaporator, a reactor with a falling-film evaporator, and/or a reactor with an attached condenser.

In another particularly preferred embodiment, a process as contemplated herein is exemplified by:
(1) mixing one or more organic C₁-C₆ alkoxy silanes with water in a reaction vessel or reactor, preferably in a double-wall reactor, a reactor with external heat exchanger, a tubular reactor, a reactor with thin-film evaporator, a reactor with falling-film evaporator and/or a reactor with attached condenser.

A reaction vessel that is very suitable for smaller preparations is, for example, a glass flask commonly used for chemical reactions with a capacity of 1 liter, 3 liters or 5 liters, such as a 3-liter single-neck or multi-neck flask with ground joints.

A reactor is a confined space (container, vessel) that has been specially designed and manufactured to allow certain reactions to take place and be controlled under defined conditions.

For larger approaches, it has proven advantageous to carry out the reaction in reactors made of metal. Typical reactors may include, for example, a 10-liter, 20-liter, or 50-liter capacity. Larger reactors for the production area can also include fill volumes of 100-liters, 500-liters, or 1000-liters.

Double-wall reactors have two reactor shells or reactor walls, with a tempering fluid circulating in the area between the two walls. This enables particularly good adjustment of the temperature to the required values.

The use of reactors, in particular double-walled reactors with an enlarged heat exchange surface, has also proven to be particularly suitable, whereby the heat exchange can take place either through internal installations or using an external heat exchanger.

Corresponding reactors are, for example, laboratory reactors from the company IKA. In this context, the models “LR-2.ST” or the model “magic plant” can be mentioned.

Other reactors that can be used are reactors with thin-film evaporators, since this allows particularly good heat dissipation and thus particularly precise temperature control. Thin film evaporators are alternatively referred to as thin film evaporators. Thin film evaporators can be purchased commercially from Asahi Glassplant Inc. for example.

In reactors with falling film evaporators, evaporation generally takes place in a tube, i.e., the liquid to be evaporated (i.e., in this case, the C₁-C₆ alcohols to be removed in step (2)) flow as a continuous liquid film. Reactors with falling film evaporators are also commercially available from various suppliers.

The reaction of the organic C₁-C₆ alkoxy silanes with water, which takes place in step (1), can occur in different ways. The reaction starts as soon as the C₁-C₆ alkoxy silanes meet water by mixing. One possibility is to place the desired amount of water in the reaction vessel or reactor and then add that or the C₁-C₆ alkoxy silanes.

In a further embodiment, it is also possible to first introduce the organic C₁-C₆ alkoxy silane(s) into the reaction vessel or reactor and then add the desired amount of water.

As soon as C₁-C₆ alkoxy silanes and water come into contact, an exothermic hydrolysis reaction takes place according to the following scheme (reaction scheme using the example of 3-aminopropyltriethoxysilane):

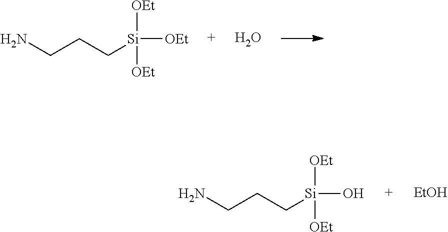 [SMILES: "EtOH" ; CCO[Si](CCCN)(OCC)OCC ; O ; CCO[Si](O)(CCCN)OCC]

Depending on the number of hydrolyzable C₁-C₆ alkoxy groups per silane molecule, the hydrolysis reaction can also occur several times per C₁-C₆ alkoxy silane used:

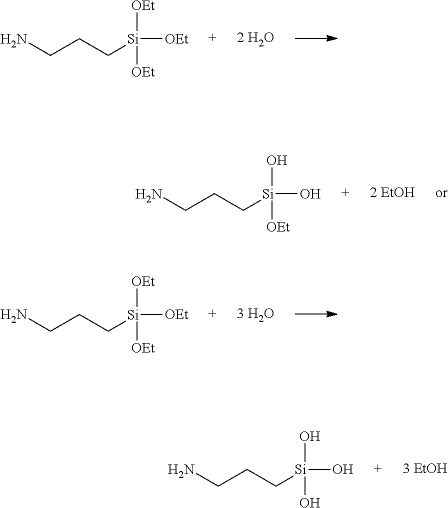

Since the hydrolysis reaction is exothermic, it has been found to be particularly advantageous to stir or mix the reaction mixture of water and organic C₁-C₆ alkoxy silanes for improved heat dissipation.

The water can be added continuously, in partial quantities or directly as a total quantity. To ensure the required temperature control, the reaction mixture is preferably cooled and/or the amount and rate of water added is adjusted. Continuous water addition is particularly preferred. Depending on the amount of silanes used, the addition and reaction can take place over a period of 2 minutes to 72 hours.

For the preparation of agents that produce a particularly good coating on the keratin material, it has been found to be explicitly quite preferred to use water in a sub-stoichiometric amount in step (1). In this case, the amount of water used is below the amount that would theoretically be required to hydrolyze all the hydrolyzable C₁-C₆ alkoxy groups present on the Si atoms, i.e., the alkoxysilane groups. Partial hydrolysis of the organic C₁-C₆ alkoxy silanes is therefore particularly preferred.

The stoichiometric ratio of water to the organic C₁-C₆ alkoxy silanes can be defined by the amount of substance equivalent water (S-W), these are calculated according to the following formula:

$$S \text{-} W = \frac{mol ( Water )}{mol ( Silane ) \times n ( Alkoxy )}$$

with

S-W=Mass equivalent water

mol(water)=molar quantity of water used

mol(silanes)=total molar amount of C₁-C₆ alkoxy silanes used in the reaction

n(alkoxy)=number of C₁-C₆ alkoxy groups per C₁-C₆ alkoxy silane

In other words, the molar equivalent of water is the molar ratio of the molar amount of water used to the total molar number of hydrolyzable C₁-C₆ alkoxy groups present on the C₁-C₆ alkoxysilanes used.

In another very particularly preferred embodiment, a method as contemplated herein is exemplified by the

(1) mixing the organic C₁-C₆ alkoxy silanes with from 0.10 to 0.80 molar equivalents of water (S-W), preferably from 0.15 to 0.70, more preferably from 0.20 to 0.60, and most preferably from 0.25 to 0.50 molar equivalents of water,

where the mass equivalents of water are calculated according to the formula

$$S \text{-} W = \frac{mol ( Water )}{mol ( Silane ) \times n ( Alkoxy )}$$

with

S-W=Mass equivalent water

mol(water)=molar quantity of water used

mol(silanes)=total molar amount of C₁-C₆ alkoxy silanes used in the reaction

n(alkoxy)=number of C₁-C₆ alkoxy groups per C₁-C₆ alkoxy silane

### EXAMPLE

In a reaction vessel, 20.0 g of 3-aminopropyltriethoxsilane (C₉H₂₃NO₃Si=221.37 g/mol) and 50.0 g of methyltrimethoxysilane (C₄H₁₂O₃Si=136.22 g/mol) were mixed.

20.0 g 3-aminopropyltriethoxsilane=0.0903 mol (3 hydrolyzable alkoxy groups per molecule)

50.0 g methyltrimethoxysilane=0.367 mol (3 hydrolyzable alkoxy groups per molecule)

Then, 10.0 g of water (18.015 g/mol) was added with stirring.

10.0 g water=0.555 mol

Mass equivalent water=0.555 mol [(3×0.090 mol)+(3×0.367 mol)]=0.40

In this reaction, the C₁-C₆ alkoxysilanes used were reacted with 0.40 molar equivalents of water.

To produce particularly high-performance keratin treatment agents, maintaining specific temperature ranges has proven to be quite advantageous in step (1).

In this context, it was found that a minimum temperature of 20° C. in step (1) is particularly well suited to allow the hydrolysis to proceed at a sufficiently high rate and to ensure efficient reaction control.

On the other hand, however, heating of the reaction mixture to temperatures above 70° C. should be avoided. If the production is carried out at too high temperatures, an undesirable or excessive polymerization or condensation reaction will probably take place at this point, resulting in the inability to form a film adhering to the keratin material during subsequent application of the agent. When using an agent produced at too high temperatures in a dyeing process, it was therefore no longer possible to achieve sufficiently high color intensities.

For these reasons, the reaction of the C₁-C₆ organic alkoxy silane(s) with water in step (1) of the process should be carried out at a temperature of 20 to 70° C.

The temperature range given here refers to the temperature to which the mixture of C₁-C₆ alkoxy silanes and water must be adjusted. This temperature can be measured, for example, by a calibrated thermometer protruding into this mixture. Preferably, the reaction of one or more organic C₁-C₆ alkoxy silanes with water occurs at a temperature of from 20° C. to 70° C., preferably from 20 to 65° C., more preferably from 20 to 60° C., still more preferably from 20 to 55° C., still more preferably from 20 to 50° C., and most preferably from 20 to 45° C.

In another very particularly preferred embodiment, a method as contemplated herein is exemplified by the

(1) mixing one or more organic C₁-C₆ alkoxy silanes with water at a temperature of from 20° C. to 70° C., preferably from 20 to 65° C., more preferably from 20 to 60° C., still more preferably from 20 to 55° C., still more preferably from 20 to 50° C., and most preferably from 20 to 45° C.

Adjustment of the preferred and particularly preferred temperature ranges can be accomplished by tempering the reaction vessel or reactor. For example, the reaction vessel or reactor may be surrounded from the outside by a temperature control bath, which may be a water bath or silicone oil bath, for example.

If the reaction is carried out in a double-walled reactor, a temperature-controlled liquid can also be passed through the space formed by the two walls surrounding the reaction chamber.

It may be further preferred that there is no active heating of the reaction mixture and that any increase in temperature above ambient is caused only by the exotherm of the hydrolysis in step (1). If the exothermic reaction process heats the reaction mixture in step (1) too much, it must be cooled again.

The reaction of the organic C₁-C₆ alkoxy silanes with water preferably takes place at normal pressure, i.e., at a pressure of 1013 mbar (1013 hPa).

### Addition of One or More Cosmetic Ingredients in Step (2).

As an optional step (2), the process as contemplated herein comprises the addition of one or more cosmetic ingredients to the mixture obtained in step (1).

The cosmetic ingredients that may optionally be used in step (2) may be any suitable ingredients to impart further beneficial properties to the product. For example, in step (2) of the process, a solvent, a thickening or film-forming polymer, a surfactant compound selected from the group including nonionic, cationic, anionic, or zwitterionic/amphoteric surfactants, colorant compounds selected from the group including pigments, direct dyes, oxidation dye precursors, fatty components from the group of C₈-C₃₀ fatty alcohols, hydrocarbon compounds, fatty acid esters, acids and bases belonging to the group of pH regulators, perfumes, preservatives, plant extracts and protein hydrolysates.

In another very particularly preferred embodiment, a method as contemplated herein is exemplified by the

(2) addition of one or more cosmetic ingredients selected from the group including solvents, polymers, surface-active compounds, coloring compounds, lipid components, pH regulators, perfumes, preservatives, plant extracts and protein hydrolysates.

The selection of these other substances will be made by the specialist according to the desired properties of the agents. Regarding other optional components and the quantities of these components used, explicit reference is made to the relevant manuals known to the specialist.

In this context, it has proven to be particularly preferred to use a cosmetic ingredient in step (2) which further improves the stability, in particular the storage stability, of the keratin treatment agent. In this context, the addition (3) of one or more cosmetic ingredients selected from the group including hexamethyldisiloxane, octamethyltrisiloxane, decamethyltetrasiloxane, hexamethylcyclotrisiloxane, octamethylcyclotetrasiloxane and/or decamethylcyclopentasiloxane has been shown to be particularly beneficial in terms of increasing the stability of the composition.

In another very particularly preferred embodiment, a method as contemplated herein is exemplified by the

(2) addition of one or more cosmetic ingredients selected from the group including hexamethyldisiloxane, octamethyltrisiloxane, decamethyltetrasiloxane, hexamethylcyclotrisiloxane, octamethylcyclotetrasiloxane and/or decamethylcyclopentasiloxane.
Hexamethyldisiloxane has the CAS number 107-46-0 and can be purchased commercially from Sigma-Aldrich, for example.

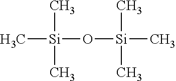

Octamethyltrisiloxane has the CAS number 107-51-7 and is also commercially available from Sigma-Aldrich.

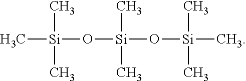

Decamethyltetrasiloxane carries the CAS number 141-62-8 and is also commercially available from Sigma-Aldrich.

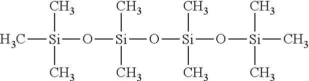

### Hexamethylcyclotrisiloxane has the CAS No. 541-05-9.

### Octamethylcyclotetrasiloxane has the CAS No. 556-67-2.

### Decamethylcyclopentasiloxane has the CAS No. 541-02-6.

### Storage of the Preparation in the Packaging Unit in Step (3).

Step (3) of the process as contemplated herein is exemplified by the storage of the mixture obtained according to step (1) or according to step (2) for a period of at least 5 days. This step has proven to be essential to some of the embodiments contemplated herein for achieving particularly intense color results.

In one embodiment, the mixture may be stored in the reaction vessel or reactor in which the mixing of the organic C₁-C₆ alkoxy silanes with water was also carried out.

However, it is preferred to fill the mixture obtained after step (1) or after step (2) into a packaging unit for the purpose of storage. This packaging may particularly preferably be a canister or a hobbock.

In the context of this embodiment, therefore, very particularly preferred is a process for preparing an agent for treating keratinous material, in particular human hair, comprising the following steps:
(1) mixing one or more organic C₁-C₆ alkoxy silanes with water,
(2) if necessary, addition of one or more cosmetic ingredients to the mixture,
(3a) filling the mixture into a packaging unit,
(3b) storage of the mixture in the packaging unit for a period of at least 5 days,
(4) if necessary, addition of one or more cosmetic ingredients to the stored mixture,
(5) partial or complete removal from the mixture of the C₁-C₆ alcohols released in steps (1) to (4).

In step (3)—or, in the case of prior filling into a packaging unit, in step (3b)—of the process as contemplated herein, the mixture or the preparation is stored for a period of at least 5 days.

If storage takes place in the reaction vessel or reactor, the mixture is allowed to stand in the sealed reaction vessel or reactor for a period of at least 5 days.

If storage takes place in a packaging unit, the packaging unit is in a sealed state during storage. This can be done, for example, by placing the sealed packaging units in a storage room or warehouse for at least 5 days.

For the purposes of the present disclosure, storage of the mixture or preparation in the reaction vessel or packaging unit is understood to mean not opening the sealed reaction vessel or packaging unit for a period of at least 5 days. Since the preparation is in a sealed container during storage, it does not meet the humidity outside the packaging unit or with oxygen.

The sealed packaging unit may be, for example, a canister or a hobbock, each sealed with a suitable lid. These usually have a larger capacity of 1 liter, 5 liters, 10 liters, 20 liters or even 50 liters. Furthermore, the packaging units that are usually used in the field of cosmetics can also be packaging made of the usual materials. These packaging units are known to the skilled person and are commercially available.

In the context of this embodiment, therefore, very particularly preferred is a process for preparing an agent for treating keratinous material, in particular human hair, comprising the following steps:

(1) mixing one or more organic C₁-C₆ alkoxy silanes with water,
(2) if necessary, addition of one or more cosmetic ingredients to the mixture,
(3a) filling the mixture into a packaging unit,
(3b) storage of the mixture in the sealed packaging unit for a period of at least 5 days,
(4) if necessary, addition of one or more cosmetic ingredients to the stored mixture,
(5) partial or complete removal from the mixture of the C₁-C₆ alcohols released in steps (1) to (4).

Without being committed to this theory, it is assumed in this context that the hydrolysis reactions initiated by mixing the C₁-C₆ alkoxy silanes with water (1) are not yet completed at the end of step (1) but continue for a period of several days thereafter. It is assumed that several days will pass before complete hydrolysis occurs, i.e., C₁-C₆ alcohols are still slowly and continuously released during at least 5 days of storage in step (3) (or in step (3b)).

In the course of the work leading to the present disclosure, it was found that good and intense colorations could be obtained when the preparations were used in a dyeing process, particularly when there was a storage period of at least 5 days between the mixing of the organic C₁-C₆ alkoxy silanes with water and the removal of the C₁-C₆ alcohols.

It has further been found to be particularly preferred to store the mixtures for a period of at least 8 days, preferably at least 10 days, more preferably at least 14 days, and most preferably at least 21 days.

In another very particularly preferred embodiment, a method as contemplated herein is exemplified by the

(3) storage of the mixture for a period of at least 8 days, preferably at least 10 days, more preferably at least 14 days, and most preferably at least 21 days.

As described previously, one theory is that oligomeric silane condensates of certain minimum size must be present to form a film on the keratin material with sufficient rapidity. On the other hand, however, the molecular weight of these silane condensates should not be too large, since good adhesion between silanes and keratin is no longer possible with condensates that are too large. Since the condensation reaction taking place during storage appears to be dependent on temperature, just like the reactions in step (1) of the process as contemplated herein, storage is also very preferably carried out within certain temperature ranges. In this context, it has proved particularly advantageous to maintain specific temperature ranges during the storage period, which takes place directly after step (1) or directly after steps (1) and (2). Particularly good dyeing results were obtained especially when the preparation was stored in the packaging unit at a temperature of 15° C. to 45° C., preferably 20° C. to 45° C., and particularly preferably 25° C. to 45° C.

In another very particularly preferred embodiment, a method as contemplated herein is exemplified by the

(3) Storing the mixture for a period of at least 8 days, preferably for at least 10 days, more preferably for at least 14 days, and most preferably for at least 21 days at a temperature in the range of from 15° C. to 45° C., preferably from 20° C. to 45° C., and most preferably from 25° C. to 45° C.

In another very particularly preferred embodiment, a method as contemplated herein is exemplified by the

3b) storing the mixture in the sealed packaging unit for a period of at least 8 days, preferably for at least 10 days, more preferably for at least 14 days, and most preferably for at least 21 days at a temperature in the range of from 15° C. to 45° C., preferably from 20° C. to 45° C., and most preferably from 25° C. to 45° C.

Particularly preferred is a process for the preparation and use of an agent for the treatment of keratinous material, in particular human hair, comprising the following steps:

(1) mixing one or more organic C₁-C₆ alkoxy silanes with water,
(2) optionally adding one or more cosmetic ingredients to the mixture obtained in step (1),
(3) Store the mixture for a period of at least 5 days at 15° C. to 45° C.,
(4) if necessary, addition of one or more cosmetic ingredients to the stored mixture,
(5) partial or complete removal from the mixture of the C₁-C₆ alcohols released in steps (1) to (4).

Treatment of keratinous material, in particular human hair, comprising the following steps:

(1) mixing one or more organic C₁-C₆ alkoxy silanes with water,
(2) optionally adding one or more cosmetic ingredients to the mixture obtained in step (1),
(3) Store the mixture for a period of at least 5 days at 20° C. to 45° C.,
(4) if necessary, addition of one or more cosmetic ingredients to the stored mixture,
(5) partial or complete removal from the mixture of the C₁-C₆ alcohols released in steps (1) to (4).

Treatment of keratinous material, in particular human hair, comprising the following steps:

(1) mixing one or more organic C₁-C₆ alkoxy silanes with water,
(2) optionally adding one or more cosmetic ingredients to the mixture obtained in step (1),
(3) store the mixture for a period of at least 5 days at 25° C. to 45° C.,
(4) if necessary, addition of one or more cosmetic ingredients to the stored mixture,
(5) partial or complete removal from the mixture of the C₁-C₆ alcohols released in steps (1) to (4).

Particularly preferred is a process for the preparation and use of an agent for the treatment of keratinous material, in particular human hair, comprising the following steps:

(1) mixing one or more organic C₁-C₆ alkoxy silanes with water,
(2) optionally adding one or more cosmetic ingredients to the mixture obtained in step (1),
(3) store the mixture for a period of at least 14 days at 15° C. to 45° C.,
(4) if necessary, addition of one or more cosmetic ingredients to the stored mixture,
(5) partial or complete removal from the mixture of the C₁-C₆ alcohols released in steps (1) to (4).

Treatment of keratinous material, in particular human hair, comprising the following steps:

(1) Mixing one or more organic C₁-C₆ alkoxy silanes with water,
(2) optionally adding one or more cosmetic ingredients to the mixture obtained in step (1),
(3) Store the mixture for a period of at least 14 days at 20° C. to 45° C.,
(4) if necessary, addition of one or more cosmetic ingredients to the stored mixture,
(5) partial or complete removal from the mixture of the C₁-C₆ alcohols released in steps (1) to (4).

Treatment of keratinous material, in particular human hair, comprising the following steps:

(1) Mixing one or more organic C₁-C₆ alkoxy silanes with water,
(2) optionally adding one or more cosmetic ingredients to the mixture obtained in step (1),
(3) Store the mixture for a period of at least 14 days at 25° C. to 45° C.,
(4) if necessary, addition of one or more cosmetic ingredients to the stored mixture,
(5) partial or complete removal from the mixture of the C₁-C₆ alcohols released in steps (1) to (4).

Particularly preferred is a process for the preparation and use of an agent for the treatment of keratinous material, in particular human hair, comprising the following steps:

(1) mixing one or more organic C₁-C₆ alkoxy silanes with water,
(2) optionally adding one or more cosmetic ingredients to the mixture obtained in step (1),
(3) store the mixture for a period of at least 21 days at 15° C. to 45° C.,
(4) if necessary, addition of one or more cosmetic ingredients to the stored mixture,
(5) partial or complete removal from the mixture of the C₁-C₆ alcohols released in steps (1) to (4).

Treatment of keratinous material, in particular human hair, comprising the following steps:

(1) mixing one or more organic C₁-C₆ alkoxy silanes with water,
(2) optionally adding one or more cosmetic ingredients to the mixture obtained in step (1),
(3) store the mixture for a period of at least 21 days at 20° C. to 45° C.,
(4) if necessary, addition of one or more cosmetic ingredients to the stored mixture,
(5) partial or complete removal from the mixture of the C₁-C₆ alcohols released in steps (1) to (4).

Treatment of keratinous material, in particular human hair, comprising the following steps:

(1) mixing one or more organic C₁-C₆ alkoxy silanes with water,
(2) optionally adding one or more cosmetic ingredients to the mixture obtained in step (1),
(3) store the mixture for a period of at least 21 days at 25° C. to 45° C.,
(4) if necessary, addition of one or more cosmetic ingredients to the stored mixture,
(5) partial or complete removal from the mixture of the C₁-C₆ alcohols released in steps (1) to (4).

Under the given storage conditions, especially within the temperature ranges, the condensation reaction of the silanes seems to come to a standstill after some time, so that a longer storage does not show any negative influence on a later dyeing result. For example, the preparations can be stored in the sealed packaging unit for a period of up to 365 days at a temperature of 15 to 40° C. Since the packaging unit is sealed during storage, thus preventing contact with the outside air, which may be humid, longer storage periods than 365 days are also possible.

In another very particularly preferred embodiment, a method as contemplated herein is exemplified by the

(3) Storage of the mixture for a period of from 5 to 365 days, preferably from 14 to 180 days, more preferably from 14 to 90 days, and most preferably from 21 to 40 days.

In another very particularly preferred embodiment, a method as contemplated herein is exemplified by the (3b) Storage of the mixture in the sealed packaging unit for a period of from 5 to 365 days, preferably from 14 to 180 days, more preferably from 14 to 90 days, and most preferably from 21 to 40 days.

### Addition of One or More Cosmetic Ingredients to the Stored Mixture (4).

In step (4) of the process as contemplated herein, one or more cosmetic ingredients may optionally be added again to the mixture. The cosmetic ingredients that can be used optionally in step (4) may again be any suitable ingredients to impart further beneficial properties to the product. For example, also in step (4) of the process, a solvent, a thickening or film-forming polymer, a surface-active compound from the group of nonionic, cationic, anionic or zwitterionic/amphoteric surfactants, the coloring compounds from the group of pigments, direct dyes, oxidation dye precursors, fatty components from the group of C₈-C₃₀ fatty alcohols, hydrocarbon compounds, fatty acid esters, acids and bases belonging to the group of pH regulators, perfumes, preservatives, plant extracts and protein hydrolysates.

In the subsequent step (5) of the process as contemplated herein, the previously released C₁-C₆ alcohols are removed as completely as possible from the mixture, this removal preferably being carried out by means of distillation under reduced pressure. To fine-tune the temperatures required for this purpose, a solvent can therefore be added to the mixture in step (4) in a particularly preferred manner, enabling a process known as “boil cooling”.

In boiling cooling, a solvent having a boiling point at normal pressure (1013 hPa) of from 20 to 90° C., preferably from 30 to 85° C. and most preferably from 40 to 80° C. is added to the reaction mixture prior to removal of the C₁-C₆ alcohols in step (5). This added solvent can also be referred to as a “low boiling point”.

The added low boiling point begins to boil at a maximum of 90° C.; at reduced pressure, the boiling temperature is lowered accordingly. If light boilers are still present in the reaction mixture, the reaction mixture is kept at the boiling temperature of the light boilers.

In another very particularly preferred embodiment, a process as contemplated herein is exemplified in that, prior to the removal of the C₁-C₆ alcohols in step (5), a solvent is added which has a boiling point at normal pressure (1013 hPa) of from 20 to 90° C., preferably from 30 to 85° C. and very particularly preferably from 40 to 80° C.

Suitable solvents include:

Dichloromethane with a boiling point of 40° C. (1013 mbar)
Methanol with a boiling point of 65° C. (1013 mbar)
Tetrahydrofuran with a boiling point of 65.8° C. (1013 mbar)
Ethanol with a boiling point of 78° C. (1013 mbar)
Isopropanol with a boiling point of 82° C. (1013 mbar)
Acetonitrile with a boiling point of 82° C. (1013 mbar)

An explicit quite particularly suitable solvent ethanol.

In a further preferred embodiment, a method as contemplated herein is exemplified by the

(4) addition of one or more cosmetic ingredients selected from the group including ethanol, isopropanol, and methanol to the stored mixture.

In another very particularly preferred embodiment, a method as contemplated herein is exemplified by the

(4) addition of ethanol to the stored mixture.

The sequence of process steps (4) and (5) can be carried out either consecutively or simultaneously. If a solvent such as ethanol is added in step (4) of the process, it may be quite preferred to perform steps (4) and (5) simultaneously. In this embodiment, ethanol is added to the stored mixture (4), and simultaneously the released C₁-C₆ alcohols are removed from the reaction mixture (5). This can be done, for example, by adding ethanol to the stored mixture while distillation is in progress under reduced pressure. This embodiment allows methanol to be removed from the reaction mixture as completely as possible.

Removal of the C₁-C₆ Alcohols Released in Step (1) from the Reaction Mixture (5)

Analytical studies suggested that C₁-C₆ organic alkoxysilanes are slowly and continuously released during the mixing of the C₁-C₆ organic alkoxysilanes with water in step (1), but also during the subsequent steps (2), (3) and (4). In step (1), when mixing the organic of the organic C₁-C₆ alkoxysilanes with water, larger amounts of C₁-C₆ alcohols are released. In addition, analytical studies have shown that significant amounts of C₁-C₆ alcohols are also formed during storage of the mixture in step (3). With a view to achieving an intensive color result in a subsequent application, it has proved particularly preferable to remove these C₁-C₆ alcohols as completely as possible from the mixture.

In another very particularly preferred embodiment, a method as contemplated herein is exemplified by the

(5) partial or complete removal from the mixture of the C₁-C₆ alcohols released by the reaction in steps (1) and (3).

Due to the removal of the C₁-C₆ alcohols, the reaction equilibrium is shifted in favor of a condensation reaction in which the Si—OH groups present on the (partially) hydrolyzed C₁-C₆ alkoxysilanes can react with further Si—OH groups or with further C₁-C₆ alkoxy-silane groups with elimination of water.

Such a reaction may proceed, for example, according to the following scheme:

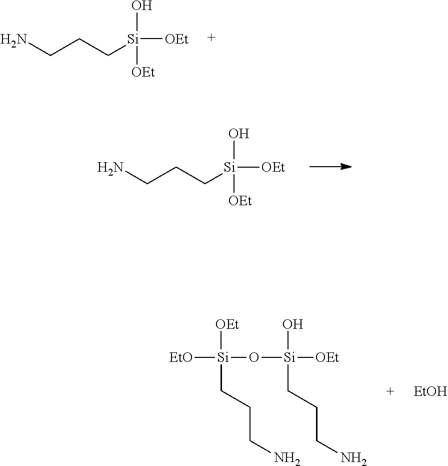

Both partially hydrolyzed and fully hydrolyzed C₁-C₆ alkoxysilanes can participate in the condensation reaction, undergoing condensation with not yet reacted, partially or also fully hydrolyzed C₁-C₆ alkoxysilanes.

In addition, condensation of C₁-C₆ alkoxysilanes of different structures is also possible; for example, the C₁-C₆ alkoxysilanes of formula (I) can condense with the C₁-C₆ alkoxysilanes of formula (IV).

In the exemplary reaction scheme above, condensation to a dimer is shown, but condensation to oligomers with multiple silane atoms is also possible and preferred.

The extent of the condensation reaction is partly determined by the amount of water added in step (1). Preferably, the amount of water is such that the condensation is a partial condensation, where “partial condensation” or “partial condensation” in this context means that not all the condensable groups of the silanes presented react with each other, so that the resulting organic silicon compound still has on average at least one hydrolyzable/condensable group per molecule.

The removal of the C₁-C₆ alcohols is preferably carried out in a reaction vessel or reactor.

If the storage in step (3) was carried out directly in this reaction vessel or reactor, the removal of the C₁-C₆ alcohols can now be carried out without further decanting.

However, if the storage in step (3) has taken place in a packaging unit such as a canister or a packaging unit, it has been found preferable to pour the mixture or preparation back into a reaction vessel or the reactor for removal of the C₁-C₆ alcohols. For example, this can again be a double-wall reactor, a reactor with external heat exchanger, a tubular reactor, a reactor with thin-film evaporator, a reactor with falling-film evaporator and/or a reactor with connected condenser. Very preferably, this reactor or reaction vessel has a distillation head for distilling off the C₁-C₆ alcohols.

Furthermore, it has been found that the temperature at which the C₁-C₆ alcohols are removed from the reaction mixture in step (5) can also be a significant influencing factor regarding the performance of the subsequent hair treatment product.

In this context, it is suspected that excessively hot temperatures above 70° C. shift condensation towards high molecular weight products that are too large to be deposited as a closed and resistant film on the keratin material during subsequent keratin treatment. For this reason, it is particularly preferred to maintain a temperature range of 20 to 70° C. when removing the C₁-C₆ alcohols from the reaction mixture.

Both complete and partial removal of the released C₁-C₆ alcohols is encompassed by the process as contemplated herein. Since the complete removal of all C₁-C₆ alcohols is difficult to realize (small residues of C₁-C₆ alcohols will always remain in the reaction mixture, especially if the reaction mixture is not to be heated too much), the partial removal of C₁-C₆ alcohols is preferred.

It is particularly preferred to maintain a temperature range of from 20° C. to 70° C., preferably from 20 to 65° C., more preferably from 20 to 60° C., still more preferably from 20 to 55° C., still more preferably from 20 to 50° C., and most preferably from 20 to 45° C. when removing the C₁-C₆ alcohols released by the reaction in step (1).

In step (5), the specified temperature range again refers to the temperature to which the reaction mixture must be adjusted while the C₁-C₆ alkoxy silanes are removed from the reaction mixture. This temperature can also be measured, for example, by a calibrated thermometer protruding into this mixture.

In another very particularly preferred embodiment, a method as contemplated herein is exemplified by the

(5) partial or complete removal of the C₁-C₆ alcohols released in steps (1) to (4) from the mixture at a temperature of from 20° C. to 70° C., preferably from 20 to 65° C., more preferably from 20 to 60° C., still more preferably from 20 to 55° C., still more preferably from 20 to 50° C. and most preferably from 20 to 45° C.

In step (5) of the process, the setting of the preferred temperature ranges as contemplated herein can be carried out, for example, by heating or cooling the reaction vessel or reactor, for example by placing the reaction vessel in a heating mantle, or by surrounding the reaction vessel from the outside with a temperature-controlled bath. The tempering bath can be, for example, a water bath or silicone oil bath.

If the reaction is carried out in a double-walled reactor, a temperature-controlled liquid can also be passed through the space formed by the two walls surrounding the reaction chamber.

In step (5) of the process, to ensure the most complete removal of the released C₁-C₆ alcohols without exceeding the essential temperature range, the C₁-C₆ alcohols are preferably removed under reduced pressure (compared to normal pressure). In this context, it has proved particularly advantageous to distill the C₁-C₆ alcohols from the reaction mixture using a distillation unit. During this distillation, a pressure of 10 to 900 mbar is preferably set, more preferably of 10 to 800 mbar, still more preferably of 10 to 600 mbar and most preferably of 10 to 300 mbar.

Vacuum distillation is a common chemical process for which standard commercially available vacuum pumps and distillation apparatus can be used. The distillation apparatus can be in the form of an attachment on the reaction vessel or reactor.

In another very particularly preferred embodiment, a method as contemplated herein is exemplified by the

(5) partial or complete removal of the C₁-C₆ alcohols liberated in steps (1) to (4) from the reaction mixture by distillation at a pressure of from 10 to 900 mbar, more preferably from 10 to 800 mbar, still more preferably from 10 to 600 mbar and very particularly preferably from 10 to 300 mbar.

Following vacuum distillation, the volatile alcohols and, if necessary, distilled water can be condensed and collected as liquid distillate in a receiver. Distillation can optionally be carried out with cooling of the evaporated alcohols/water by means of a cooler. The reduced pressure can be generated by means of common processes known in the prior art, typically with a vacuum pump.

As already described, C₁-C₆-alkoxysilanes carrying methoxysilane or ethoxysilane groups, di- and trimethoxy- and -ethoxysilanes, especially preferably trimethoxy- or triethoxysilanes, are very preferably used in the process as contemplated herein. These have the advantage that methanol and ethanol are released during hydrolysis and condensation, respectively, which can be easily removed from the reaction mixture by vacuum distillation due to their boiling points.

In various embodiments, the vacuum distillation of step (5) is carried out under conditions that yield a product containing less than 5% by weight, preferably less than 2% by weight, more preferably less than 1% by weight of alcohols (from the hydrolysis reaction). The water content of the product after vacuum distillation is less than 5.0 wt %, even more preferably less than 1.0 wt %, and most preferably less than 0.5 wt %.

With the completion of step (5), the feature for treating the keratinous material is now available.

Filling the Agent into a Packaging Unit (6)

Following step (5), the keratinous material treatment agent is preferably filled into a packaging unit.

The packaging unit can either be a final packaging from which the user takes the agent for treatment of the keratin materials. Suitable end-packages include a bottle, a tube, ajar, a can, a sachet, an aerosol pressure container, a non-aerosol pressure container. In this regard, these final packages may contain the keratin treatment agents in quantities sufficient for one, or if necessary, several applications. Preference is given to filling in a quantity sufficient for a single application.

Further, however, the preparation in step (6) may also be filled into an intermediate package, which may be, for example, a canister or a hobbock. Filling into an intermediate package is particularly suitable if the reaction vessel or reactor in which the removal of the C₁-C₆ alcohols was carried out and the filling plant in which a filling into the final package is carried out are physically separated.

In another very particularly preferred embodiment, a method as contemplated herein is exemplified by the

(6) filling the agent into a bottle, tube, jar, can, sachet, aerosol pressure container, non-aerosol pressure container, canister or hobbock.

In other words, a particularly preferred method is exemplified by the

(6) filling the mixture into a bottle, tube, jar, can, sachet, aerosol pressure container, non-aerosol pressure container, canister, or hobbock.

The packaging units may be common, standard, commercially available containers used in cosmetics.

### Sequence of the Process Steps

It is characteristic of the method as contemplated herein that it comprises steps (1), (2), (3), (4), and (5), wherein steps (2) and (4) are each optional steps. Regarding the sequence of the process steps, several embodiments are suitable.

In one embodiment, preferred is a method comprising the steps in the following order:

(1) mixing one or more organic C₁-C₆ alkoxy silanes with water,
(2) if necessary, addition of one or more cosmetic ingredients to the mixture,
(3) storage of the mixture for a period of at least 5 days,
(4) where appropriate, addition of one or more cosmetic ingredients to the stored mixture; and
(5) partial or complete removal from the mixture of the C₁-C₆ alcohols released in steps (1) to (4).

In an embodiment, a method comprising of the following steps is preferred:

(1) mixing one or more organic C₁-C₆ alkoxy silanes with water,
(2) if necessary, addition of one or more cosmetic ingredients to the mixture,
(3) storage of the mixture for a period of at least 5 days,
(4) addition of one or more cosmetic ingredients to the stored mixture, and
(5) partial or complete removal from the mixture of the C₁-C₆ alcohols released in steps (1) to (4),
wherein first step (1) is carried out, then, if necessary, step (2) is carried out, then step (3) is carried out and subsequently steps (4) and (5) are carried out simultaneously.

In the context of one embodiment, explicitly quite particularly preferred is a method comprising the steps of

(1) mixing one or more organic C₁-C₆ alkoxy silanes with water,
(2) if necessary, addition of one or more cosmetic ingredients to the mixture,
(3) storage of the mixture for a period of at least 5 days,
(4) addition of ethanol to the stored mixture, and
(5) partial or complete removal of C₁-C₆ alcohols from the mixture,
wherein first step (1) is carried out, then, if necessary, step (2) is carried out, then step (3) is carried out and subsequently steps (4) and (5) are carried out simultaneously.

Further preferred is also a process for preparing an agent for treating keratinous material, in particular human hair, comprising the following steps:

(1) mixing one or more organic C₁-C₆ alkoxy silanes with water,
(2) if necessary, addition of one or more cosmetic ingredients to the mixture,
(3) storage of the mixture for a period of at least 5 days,
(4) where appropriate, addition of one or more cosmetic ingredients to the stored mixture; and
(5) partial or complete removal from the mixture of the C₁-C₆ alcohols released in steps (1) to (4), and
(6) filling the mixture into a bottle, tube, jar, can, sachet, aerosol pressure container, non-aerosol pressure container, canister or hobbock.

### pH Values of the Preparations in the Process

In further experiments, it has been found that the pH values possessed by the mixture during steps (1) to (5) of the process as contemplated herein can also have an influence on the condensation reaction. It was found that alkaline pH values in particular stop condensation at the oligomer stage. The more acidic the reaction mixture, the stronger the condensation seems to proceed and the higher the molecular weight of the silane condensates formed during condensation. For this reason, it is preferred that the reaction mixture in step (1), (2), (3), (4) and/or (5) has a pH of from 7.0 to 12.0, preferably from 7.5 to 11.5, more preferably from 8.5 to 11.0, and most preferably from 9.0 to 11.0.

The water content of the agent produced by the process as contemplated herein is at most 10.0 wt. % and is preferably set even lower. Particularly in the case of compositions with an extremely low water content, measuring the pH with the usual methods known from the prior art (pH value measurement by means of glass electrodes via combination electrodes or via pH indicator paper) can prove difficult. For this reason, the pH values as contemplated herein are those obtained after mixing or diluting the preparation in a 1:1 ratio by weight with distilled water.

Accordingly, the corresponding pH is measured after, for example, 50 g of the composition as contemplated herein has been mixed with 50 g of distilled water.

In another very particularly preferred embodiment, a process as contemplated herein, exemplified in that the mixture in step (1), (2), (3), (4) and/or (5) and/or (6), after dilution with distilled water in a weight ratio of 1:1, has a pH of from 7.5 to 11.5, more preferably from 8.5 to 11.0 and very particularly preferably from 9.0 to 11.0.

To adjust this alkaline pH, it may be necessary to add an alkalizing agent and/or acidifying agent to the reaction mixture. The pH values for the purposes of the present disclosure are pH values measured at a temperature of 22° C.

For example, ammonia, alkanolamines and/or basic amino acids can be used as alkalizing agents.

Alkanolamines may be selected from primary amines having a C₂-C₆ alkyl parent bearing at least one hydroxyl group. Preferred alkanolamines are selected from the group formed by 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropan-1,2-diol, 2-amino-2-methylpropan-1,3-diol.

For the purposes of this disclosure, an amino acid is an organic compound containing in its structure at least one protonatable amino group and at least one —COOH or one —SO₃H group. Preferred amino acids are amino carboxylic acids, especially α-(alpha)-amino carboxylic acids and ω-amino carboxylic acids, whereby α-amino carboxylic acids are particularly preferred.

As contemplated herein, basic amino acids are those amino acids which have an isoelectric point pI of greater than 7.0.

Basic α-amino carboxylic acids contain at least one asymmetric carbon atom. In the context of the present disclosure, both possible enantiomers can be used equally as specific compounds or their mixtures, especially as racemates. However, it is particularly advantageous to use the naturally preferred isomeric form, usually in L-configuration.

The basic amino acids are preferably selected from the group formed by arginine, lysine, ornithine, and histidine, especially preferably arginine and lysine. In another particularly preferred embodiment, an agent as contemplated herein is therefore exemplified in that the alkalizing agent is a basic amino acid from the group arginine, lysine, omithine and/or histidine.

In addition, inorganic alkalizing agents can also be used. Inorganic alkalizing agents usable as contemplated herein are preferably selected from the group formed by sodium hydroxide, potassium hydroxide, calcium hydroxide, barium hydroxide, sodium phosphate, potassium phosphate, sodium silicate, sodium metasilicate, potassium silicate, sodium carbonate and potassium carbonate.

Particularly preferred alkalizing agents are ammonia, 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-Amino-2-methylpropan-2-ol, 3-aminopropan-1,2-diol, 2-amino-2-methylpropan-1,3-diol, arginine, lysine, ornithine, histidine, sodium hydroxide, potassium hydroxide, calcium hydroxide, barium hydroxide, sodium phosphate, potassium phosphate, sodium silicate, sodium metasilicate, potassium silicate, sodium carbonate and potassium carbonate.

Apart from the alkalizing agents described above, experts are familiar with common acidifying agents for fine adjustment of the pH value. As contemplated herein, preferred acidifiers are pleasure acids, such as citric acid, acetic acid, malic acid, or tartaric acid, as well as diluted mineral acids.

### Agent for the Treatment of Keratinous Material

The process described above allows the preparation of prehydrolyzed or condensed silane blends, which can be applied to the keratinous material after completion of steps (5) and (6), respectively.

In principle, the keratin treatment agents produced by this process can be used for various purposes, for example as agents for coloring keratinous material, as agents for caring for keratinous material or as agents for changing the shape of keratinous material.

In another very particularly preferred embodiment, a process as contemplated herein is exemplified in that an agent is prepared for coloring keratinous material, for maintaining keratinous material or for changing the shape of keratinous material.

In another very particularly preferred embodiment, a process as contemplated herein is exemplified in that an agent is prepared for coloring keratinous material, for maintaining keratinous material or for changing the shape of keratinous material.

Explicitly, the prepared agents show particularly good suitability when used in a dyeing process.

In another very particularly preferred embodiment, a process as contemplated herein is exemplified in that an agent for coloring keratinous material is prepared.

When used in a dyeing process, at least one colorant compound may also be added to the composition, for example in step (2) and/or step (4), wherein the colorant compound may be selected from the group including pigments, direct dyes and/or oxidation dye precursors. Here, an agent for coloring keratin material can be obtained which, in addition to the prehydrolyzed/condensed C₁-C₆ alkoxysilanes, also contains the coloring compound(s).

However, it is also preferred if the user applies the hair colorant to the keratin material as part of a multi-component packaging unit.

In this embodiment, the stored preparation is first mixed with another preparation after step (5) or after step (6), so that a ready-to-use colorant is obtained. This ready-to-use colorant is then applied to the keratin materials.

### Multi-Component Packaging Unit (Kit-of-Parts)

To increase user convenience, all preparations required for the staining process are provided to the user in the form of a multi-component packaging unit (kit-of-parts).

A second object of the present disclosure is a multi-component packaging unit (kit-of-parts) for dyeing keratinous material, in particular human hair, which separately includes

a first packaging unit containing a cosmetic preparation (A) and
a second packaging unit containing a cosmetic preparation (B),
where
the cosmetic preparation (A) in the first packaging unit is an agent which, in accordance with the steps (1) to (5) of the process disclosed in detail in the description of the present disclosure, and
the cosmetic formulation (B) comprises at least one colorant compound selected from the group including pigments, direct dyes and/or oxidation dye precursors.

Just before application, the two preparations (A) and (B) are then mixed, and this ready-to-use staining agent is then applied to the keratin material.

Furthermore, the multi-component packaging unit as contemplated herein may also comprise a third packaging unit containing a cosmetic preparation (C). Preparation (C) may be, for example, a conditioner, a shampoo, or a pre- or post-treatment agent.

### Coloring Compounds

When the agents prepared via the process as contemplated herein are used in a dyeing process, one or more colorant compounds may be employed. The colorant compound(s) can either be added to the reaction mixture as cosmetic ingredients in step (3) of the process or provided to the user as an ingredient of a separately prepared preparation (B).

The coloring compound or compounds can preferably be selected from pigments, substantive dyes, oxidation dyes, photochromic dyes and thermochromic dyes, particularly preferably from pigments and/or substantive dyes.

Pigments within the meaning of the present disclosure are coloring compounds which have a solubility in water at 25° C. of less than 0.5 g/L, preferably less than 0.1 g/L, even more preferably less than 0.05 g/L. Water solubility can be determined, for example, by the method described below: 0.5 g of the pigment are weighed in a beaker. A stir-fish is added. Then one liter of distilled water is added. This mixture is heated to 25° C. for one hour while stirring on a magnetic stirrer. If undissolved components of the pigment are still visible in the mixture after this period, the solubility of the pigment is below 0.5 g/L. If the pigment-water mixture cannot be assessed visually due to the high intensity of the possibly finely dispersed pigment, the mixture is filtered. If a proportion of undissolved pigments remains on the filter paper, the solubility of the pigment is below 0.5 g/L.

Suitable color pigments can be of inorganic and/or organic origin.

In a preferred embodiment, a composition as contemplated herein comprises at least one colorant compound selected from the group including inorganic and/or organic pigments.

Preferred color pigments are selected from synthetic or natural inorganic pigments. Inorganic color pigments of natural origin can be produced, for example, from chalk, ochre, umber, green earth, burnt Terra di Siena or graphite. Furthermore, black pigments such as iron oxide black, colored pigments such as ultramarine or iron oxide red as well as fluorescent or phosphorescent pigments can be used as inorganic color pigments.

Particularly suitable are colored metal oxides, hydroxides and oxide hydrates, mixed-phase pigments, sulfur-containing silicates, silicates, metal sulfides, complex metal cyanides, metal sulphates, chromates and/or molybdates. Preferred color pigments are black iron oxide (CI 77499), yellow iron oxide (CI 77492), red and brown iron oxide (CI 77491), manganese violet (CI 77742), ultramarine (sodium aluminum sulfo silicates, CI 77007, pigment blue 29), chromium oxide hydrate (CI77289), iron blue (ferric ferrocyanides, C177510) and/or carmine (cochineal).

Colored pearlescent pigments are also particularly preferred colorants from the group of pigments as contemplated herein. These are usually mica- and/or mica-based and can be coated with one or more metal oxides. Mica belongs to the layer silicates. The most important representatives of these silicates are muscovite, phlogopite, paragonite, biotite, lepidolite and margarite. To produce the pearlescent pigments in combination with metal oxides, the mica, mainly muscovite or phlogopite, is coated with a metal oxide.

As an alternative to natural mica, synthetic mica coated with one or more metal oxides can also be used as pearlescent pigment. Especially preferred pearlescent pigments are based on natural or synthetic mica (mica) and are coated with one or more of the metal oxides mentioned above. The color of the respective pigments can be varied by varying the layer thickness of the metal oxide(s).

In a further preferred embodiment, an agent as contemplated herein comprises (b) at least one colorant compound from the group of pigments selected from the group of colored metal oxides, metal hydroxides, metal oxide hydrates, silicates, metal sulfides, complex metal cyanides, metal sulfates, bronze pigments and/or from mica- or mica-based colorant compounds coated with at least one metal oxide and/or a metal oxychloride.

In a further preferred embodiment, a composition as contemplated herein comprises (b) at least one colorant compound selected from mica- or mica-based pigments reacted with one or more metal oxides selected from the group including titanium dioxide (CI 77891), black iron oxide (CI 77499), yellow iron oxide (CI 77492), red and/or brown iron oxide (CI 77491, CI 77499), manganese violet (CI 77742), ultramarines (sodium aluminum sulfosilicates, CI 77007, Pigment Blue 29), chromium oxide hydrate (CI 77289), chromium oxide (CI 77288) and/or iron blue (ferric ferrocyanide, CI 77510).

Examples of particularly suitable color pigments are commercially available under the trade names Rona®, Colorona®, Xirona®, Dichrona® and Timiron® from Merck, Ariabel® and Unipure® from Sensient, Prestige® from Eckart Cosmetic Colors and Sunshine® from Sunstar.

Particularly preferred color pigments with the trade name Colorona® are, for example:

### Colorona Copper, Merck, MICA, CI 77491 (IRON OXIDES)

### Colorona Passion Orange, Merck, Mica, CI 77491 (Iron Oxides), Alumina

### Colorona Patina Silver, Merck, MICA, CI 77499 (IRON OXIDES), CI 77891 (TITANIUM DIOXIDE)

### Colorona RY, Merck, CI 77891 (TITANIUM DIOXIDE), MICA, CI 75470 (CARMINE)

### Colorona Oriental Beige, Merck, MICA, CI 77891 (TITANIUM DIOXIDE), CI 77491 (IRON OXIDES)

### Colorona Dark Blue, Merck, MICA, TITANIUM DIOXIDE, FERRIC FERROCYANIDE

### Colorona Chameleon, Merck, CI 77491 (IRON OXIDES), MICA

### Colorona Aborigine Amber, Merck, MICA, CI 77499 (IRON OXIDES), CI 77891 (TITANIUM DIOXIDE)

### Colorona Blackstar Blue, Merck, CI 77499 (IRON OXIDES), MICA

### Colorona Patagonian Purple, Merck, MICA, CI 77491 (IRON OXIDES), CI 77891 (TITANIUM DIOXIDE), CI 77510 (FERRIC FERROCYANIDE)

### Colorona Red Brown, Merck, MICA, CI 77491 (IRON OXIDES), CI 77891 (TITANIUM DIOXIDE)

### Colorona Russet, Merck, CI 77491 (TITANIUM DIOXIDE), MICA, CI 77891 (IRON OXIDES)

### Colorona Imperial Red, Merck, MICA, TITANIUM DIOXIDE (CI 77891), D&C RED NO. 30 (CI 73360)

### Colorona Majestic Green, Merck, CI 77891 (TITANIUM DIOXIDE), MICA, CI 77288 (CHROMIUM OXIDE GREENS)

### Colorona Light Blue, Merck, MICA, TITANIUM DIOXIDE (CI 77891), FERRIC FERROCYANIDE (CI 77510)

### Colorona Red Gold, Merck, MICA, CI 77891 (TITANIUM DIOXIDE), CI 77491 (IRON OXIDES)

### Colorona Gold Plus MP 25, Merck, MICA, TITANIUM DIOXIDE (CI 77891), IRON OXIDES (CI 77491)

### Colorona Carmine Red, Merck, MICA, TITANIUM DIOXIDE, CARMINE

### Colorona Blackstar Green, Merck, MICA, CI 77499 (IRON OXIDES)

### Colorona Bordeaux, Merck, MICA, CI 77491 (IRON OXIDES)

### Colorona Bronze, Merck, MICA, CI 77491 (IRON OXIDES)

### Colorona Bronze Fine, Merck, MICA, CI 77491 (IRON OXIDES)

### Colorona Fine Gold MP 20, Merck, MICA, CI 77891 (TITANIUM DIOXIDE), CI 77491 (IRON OXIDES)

### Colorona Sienna Fine, Merck, CI 77491 (IRON OXIDES), MICA

### Colorona Sienna, Merck, MICA, CI 77491 (IRON OXIDES)

Colorona Precious Gold, Merck, Mica, CI 77891 (Titanium dioxide), Silica, CI 77491 (Iron oxides), Tin oxide

### Colorona Sun Gold Sparkle MP 29, Merck, MICA, TITANIUM DIOXIDE, IRON OXIDES, MICA, CI 77891, CI 77491 (EU)

Colorona Mica Black, Merck, CI 77499 (Iron oxides), Mica, CI 77891 (Titanium dioxide)
Colorona Bright Gold, Merck, Mica, CI 77891 (Titanium dioxide), CI 77491 (Iron oxides)

### Colorona Blackstar Gold, Merck, MICA, CI 77499 (IRON OXIDES)

Other particularly preferred color pigments with the trade name Xirona® are for example:

### Xirona Golden Sky, Merck, Silica, CI 77891 (Titanium Dioxide), Tin Oxide

### Xirona Caribbean Blue, Merck, Mica, CI 77891 (Titanium Dioxide), Silica, Tin Oxide

### Xirona Kiwi Rose, Merck, Silica, CI 77891 (Titanium Dioxide), Tin Oxide

### Xirona Magic Mauve, Merck, Silica, CI 77891 (Titanium Dioxide), Tin Oxide.

In addition, particularly preferred color pigments with the trade name Unipure® are for example:

### Unipure Red LC 381 EM, Sensient CI 77491 (Iron Oxides), Silica

### Unipure Black LC 989 EM, Sensient, CI 77499 (Iron Oxides), Silica

### Unipure Yellow LC 182 EM, Sensient, CI 77492 (Iron Oxides), Silica

In a further embodiment, the composition or preparation as contemplated herein may also contain one or more colorant compounds selected from the group including organic pigments

The organic pigments as contemplated herein are correspondingly insoluble, organic dyes or color lacquers, which may be selected, for example, from the group of nitroso, nitro-azo, xanthene, anthraquinone, isoindolinone, isoindolinone, quinacridone, perinone, perylene, diketo-pyrrolopyorrole, indigo, thioindido, dioxazine and/or triarylmethane compounds.

Examples of particularly suitable organic pigments are carmine, quinacridone, phthalocyanine, sorghum, blue pigments with the Color Index numbers Cl 42090, CI 69800, CI 69825, CI 73000, CI 74100, CI 74160, yellow pigments with the Color Index numbers CI 11680, CI 11710, CI 15985, CI 19140, CI 20040, CI 21100, CI 21108, CI 47000, CI 47005, green pigments with the Color Index numbers CI 61565, CI 61570, CI 74260, orange pigments with the Color Index numbers CI 11725, CI 15510, CI 45370, CI 71105, red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

In a further particularly preferred embodiment, a composition as contemplated herein comprises at least one colorant compound from the group of organic pigments selected from the group including carmine, quinacridone, phthalocyanine, sorghum, blue pigments having the Color Index numbers Cl 42090, CI 69800, CI 69825, CI 73000, CI 74100, CI 74160, yellow pigments having the Color Index numbers CI 11680, CI 11710, CI 15985, CI 19140, CI 20040, CI 21100, CI 21108, CI 47000, CI 47005, green pigments with Color Index numbers CI 61565, CI 61570, CI 74260, orange pigments with Color Index numbers CI 11725, CI 15510, CI 45370, CI 71105, red pigments with Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

The organic pigment can also be a color paint. As contemplated herein, the term color lacquer means particles comprising a layer of absorbed dyes, the unit of particle and dye being insoluble under the above-mentioned conditions. The particles can, for example, be inorganic substrates, which can be aluminum, silica, calcium borosilate, calcium aluminum borosilicate or even aluminum. For example, alizarin color varnish can be used.

Due to their excellent resistance to light and temperature, the use of the pigments in the means as contemplated herein is particularly preferred. It is also preferred if the pigments used have a certain particle size. This particle size leads on the one hand to an even distribution of the pigments in the formed polymer film and on the other hand avoids a rough hair or skin feeling after application of the cosmetic product. As contemplated herein, it is therefore advantageous if the at least one pigment has an average particle size D₅₀ of 1.0 to 50 μm, preferably 5.0 to 45 μm, preferably 10 to 40 μm, 14 to 30 μm. The mean particle size D50D₅₀, for example, can be determined using dynamic light scattering (DLS).

The pigment or pigments may be used in an amount of from 0.001 to 20% by weight, from 0.05 to 5% by weight, in each case based on the total weight of the composition or preparation as contemplated herein.

As colorant compounds, the compositions as contemplated herein may also contain one or more direct dyes. Direct-acting dyes are dyes that draw directly onto the hair and do not require an oxidative process to form the color. Direct dyes are usually nitrophenylene diamines, nitroaminophenols, azo dyes, anthraquinones, triarylmethane dyes or indophenols.

The direct dyes within the meaning of the present disclosure have a solubility in water (760 mmHg) at 25° C. of more than 0.5 g/L and are therefore not to be regarded as pigments. Preferably, the direct dyes within the meaning of the present disclosure have a solubility in water (760 mmHg) at 25° C. of more than 1.0 g/L. In particular, the direct dyes within the meaning of the present disclosure have a solubility in water (760 mmHg) at 25° C. of more than 1.5 g/L.

Direct dyes can be divided into anionic, cationic, and nonionic direct dyes.

In a further preferred embodiment, an agent as contemplated herein contains at least one anionic, cationic and/or nonionic direct dye as the coloring compound.

In a further preferred embodiment, an agent as contemplated herein comprises at least one anionic, cationic and/or nonionic direct dye.

Suitable cationic direct dyes include Basic Blue 7, Basic Blue 26, Basic Violet 2, and Basic Violet 14, Basic Yellow 57, Basic Red 76, Basic Blue 16, Basic Blue 347 (Cationic Blue 347/Dystar), HC Blue No. 16, Basic Blue 99, Basic Brown 16, Basic Brown 17, Basic Yellow 57, Basic Yellow 87, Basic Orange 31, Basic Red 51 Basic Red 76

As non-ionic direct dyes, non-ionic nitro and quinone dyes and neutral azo dyes can be used. Suitable non-ionic direct dyes are those listed under the international designations or Trade names HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9 known compounds, as well as 1,4-diamino-2-nitrobenzene, 2-amino-4-nitrophenol, 1,4-bis-(2-hydroxyethyl)-amino-2-nitrobenzene, 3-nitro-4-(2-hydroxyethyl)-aminophenol 2-(2-hydroxyethyl)amino-4,6-dinitrophenol, 4-[(2-hydroxyethyl)amino]-3-nitro-1-methylbenzene, 1-amino-4-(2-hydroxyethyl)-amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 6-nitro-1,2,3,4-tetrahydroquinoxaline, 2-hydroxy-1,4-naphthoquinone, picramic acid and its salts, 2-amino-6-chloro-4-nitrophenol, 4-ethylamino-3-nitrobenzoic acid and 2-chloro-6-ethylamino-4-nitrophenol.

Anionic direct dyes are also called acid dyes. Acid dyes are direct dyes that have at least one carboxylic acid group (—COOH) and/or one sulphonic acid group (—SO₃H). Depending on the pH value, the protonated forms (—COOH, —SO₃H) of the carboxylic acid or sulphonic acid groups are in equilibrium with their deprotonated forms (—COO⁻, —SO₃⁻ present). The proportion of protonated forms increases with decreasing pH. If direct dyes are used in the form of their salts, the carboxylic acid groups or sulphonic acid groups are present in deprotonated form and are neutralized with corresponding stoichiometric equivalents of cations to maintain electro neutrality. Contemplated acid dyes can also be used in the form of their sodium salts and/or their potassium salts.

The acid dyes within the meaning of the present disclosure have a solubility in water (760 mmHg) at 25° C. of more than 0.5 g/L and are therefore not to be regarded as pigments. Preferably the acid dyes within the meaning of the present disclosure have a solubility in water (760 mmHg) at 25° C. of more than 1.0 g/L.

The alkaline earth salts (such as calcium salts and magnesium salts) or aluminum salts of acid dyes often have a lower solubility than the corresponding alkali salts. If the solubility of these salts is below 0.5 g/L (25° C., 760 mmHg), they do not fall under the definition of a direct dye.

An essential characteristic of acid dyes is their ability to form anionic charges, whereby the carboxylic acid or sulphonic acid groups responsible for this are usually linked to different chromophoric systems. Suitable chromophoric systems can be found, for example, in the structures of nitrophenylenediamines, nitroaminophenols, azo dyes, anthraquinone dyes, triarylmethane dyes, xanthene dyes, rhodamine dyes, oxazine dyes and/or indophenol dyes.

For example, one or more compounds from the following group can be selected as particularly well suited acid dyes: Acid Yellow 1 (D&C Yellow 7, Citronin A, Ext. D&C Yellow No. 7, Japan Yellow 403, CI 10316, COLIPA no B001), Acid Yellow 3 (COLIPA no: C 54, D&C Yellow No 10, Quinoline Yellow, E104, Food Yellow 13), Acid Yellow 9 (CI 13015), Acid Yellow 17 (CI 18965), Acid Yellow 23 (COLIPA no C 29, Covacap Jaune W 1100 (LCW), Sicovit Tartrazine 85 E 102 (BASF), Tartrazine, Food Yellow 4, Japan Yellow 4, FD&C Yellow No. 5), Acid Yellow 36 (CI 13065), Acid Yellow 121 (CI 18690), Acid Orange 6 (CI 14270), Acid Orange 7 (2-Naphthol orange, Orange II, CI 15510, D&C Orange 4, COLIPA no C015), Acid Orange 10 (C.I. 16230; Orange G sodium salt), Acid Orange 11 (CI 45370), Acid Orange 15 (CI 50120), Acid Orange 20 (CI 14600), Acid Orange 24 (BROWN 1; CI 20170; KATSU201; nosodiumsalt; Brown No. 201; RESORCIN BROWN; ACID ORANGE 24; Japan Brown 201; D & C Brown No. 1), Acid Red 14 (C.I.14720), Acid Red 18 (E124, Red 18; CI 16255), Acid Red 27 (E 123, CI 16185, C-Rot 46, Real red D, FD&C Red Nr.2, Food Red 9, Naphthol red S), Acid Red 33 (Red 33, Fuchsia Red, D&C Red 33, CI 17200), Acid Red 35 (CI C.I.18065), Acid Red 51 (CI 45430, Pyrosin B, Tetraiodfluorescein, Eosin J, Iodeosin), Acid Red 52 (CI 45100, Food Red 106, Solar Rhodamine B, Acid Rhodamine B, Red no 106 Pontacyl Brilliant Pink), Acid Red 73 (CI 27290), Acid Red 87 (Eosin, CI 45380), Acid Red 92 (COLIPA no C53, CI 45410), Acid Red 95 (CI 45425, Erythtosine, Simacid Erythrosine Y), Acid Red 184 (CI 15685), Acid Red 195, Acid Violet 43 (Jarocol Violet 43, Ext. D&C Violet no 2, C.I. 60730, COLIPA no C063), Acid Violet 49 (CI 42640), Acid Violet 50 (CI 50325), Acid Blue 1 (Patent Blue, CI 42045), Acid Blue 3 (Patent Blue V, CI 42051), Acid Blue 7 (CI 42080), Acid Blue 104 (CI 42735), Acid Blue 9 (E 133, Patent blue AE, Amino blue AE, Erioglaucin A, CI 42090, C.I. Food Blue 2), Acid Blue 62 (CI 62045), Acid Blue 74 (E 132, CI 73015), Acid Blue 80 (CI 61585), Acid Green 3 (CI 42085, Foodgreen1), Acid Green 5 (CI 42095), Acid Green 9 (C.I.42100), Acid Green 22 (C.I.42170), Acid Green 25 (CI 61570, Japan Green 201, D&C Green No. 5), Acid Green 50 (Brilliant Acid Green BS, C.I. 44090, Acid Brilliant Green BS, E 142), Acid Black 1 (Black no 401, Naphthalene Black 10B, Amido Black 10B, CI 20 470, COLIPA no B15), Acid Black 52 (CI 15711), Food Yellow 8 (CI 14270), Food Blue 5, D&C Yellow 8, D&C Green 5, D&C Orange 10, D&C Orange 11, D&C Red 21, D&C Red 27, D&C Red 33, D&C Violet 2 and/or D&C Brown 1.

For example, the water solubility of anionic direct dyes can be determined in the following way. 0.1 g of the anionic direct dye is placed in a beaker. A stir-fish is added. Then add 100 ml of water. This mixture is heated to 25° C. on a magnetic stirrer while stirring. It is stirred for 60 minutes. The aqueous mixture is then visually assessed. If there are still undissolved residues, the amount of water is increased—for example in steps of 10 ml. Water is added until the amount of dye used is completely dissolved. If the dye-water mixture cannot be assessed visually due to the high intensity of the dye, the mixture is filtered. If a proportion of undissolved dyes remains on the filter paper, the solubility test is repeated with a higher quantity of water. If 0.1 g of the anionic direct dye dissolves in 100 ml water at 25° C., the solubility of the dye is 1.0 g/L.

Acid Yellow 1 is called 8-hydroxy-5,7-dinitro-2-naphthalenesulfonic acid disodium salt and has a solubility in water of at least 40 g/L (25° C.).

Acid Yellow 3 is a mixture of the sodium salts of mono- and sisulfonic acids of 2-(2-quinolyl)-1H-indene-1,3(2H)-dione and has a water solubility of 20 g/L (25° C.).
Acid Yellow 9 is the disodium salt of 8-hydroxy-5,7-dinitro-2-naphthalenesulfonic acid, its solubility in water is above 40 g/L (25° C.).
Acid Yellow 23 is the trisodium salt of 4,5-dihydro-5-oxo-1-(4-sulfophenyl)-4-((4-sulfophenyl)azo)-1H-pyrazole-3-carboxylic acid and is highly soluble in water at 25° C.
Acid Orange 7 is the sodium salt of 4-[(2-hydroxy-1-naphthyl)azo]benzene sulphonate. Its water solubility is more than 7 g/L (25° C.).
Acid Red 18 is the trinatirum salt of 7-hydroxy-8-[(E)-(4-sulfonato-1-naphthyl)-diazenyl)]-1,3-naphthalene disulfonate and has a very high-water solubility of more than 20% by weight.
Acid Red 33 is the diantrium salt of 5-amino-4-hydroxy-3-(phenylazo)-naphthalene-2,7-disulphonate, its solubility in water is 2.5 g/L (25° C.).
Acid Red 92 is the disodium salt of 3,4,5,6-tetrachloro-2-(1,4,5,8-tetrabromo-6-hydroxy-3-oxoxanthen-9-yl)benzoic acid, whose solubility in water is indicated as greater than 10 g/L (25° C.).
Acid Blue 9 is the disodium salt of 2-({4-[N-ethyl(3-sulfonatobenzyl]amino]phenyl)}4-[(N-ethyl(3-sulfonatobenzyl)imino]-2,5-cyclohexadien-1-ylidene)methyl)-benzenesulfonate and has a solubility in water of more than 20% by weight (25° C.).

Thermochromic dyes can also be used. Thermochromism involves the property of a material to change its color reversibly or irreversibly as a function of temperature. This can be done by changing both the intensity and/or the wavelength maximum.

Finally, it is also possible to use photochromic dyes. Photochromism involves the property of a material to change its color depending reversibly or irreversibly on irradiation with light, especially UV light. This can be done by changing both the intensity and/or the wavelength maximum.

Also, the preparation (B) may additionally contain one or more further ingredients selected from the group of solvents, thickening or film-forming polymers, surface-active compounds from the group of nonionic, cationic, anionic or zwitterionic/amphoteric surfactants, of the fatty components from the group of C₈-C₃₀ fatty alcohols, hydrocarbon compounds, fatty acid esters, acids and bases belonging to the group of pH regulators, perfumes, preservatives, plant extracts and protein hydrolysates.

With respect to the other preferred embodiments of the multi-component packaging unit as contemplated herein, the same applies mutatis mutandis to the procedure as contemplated herein.

### EXAMPLES

### 1. Preparation of the Silane Blend

A reactor with a heatable/coolable outer shell and with a capacity of 15 liters was filled with 4.67 kg of methyltrimethoxysilane. 1.33 kg of (3-aminopropyl)triethoxysilane was then added with stirring. This mixture was stirred at 30° C. Subsequently, 670 ml of water (dist.) was added dropwise with vigorous stirring, maintaining the temperature of the reaction mixture at 30° C. under external cooling. After completion of the water addition, stirring was continued for another 10 minutes. To the mixture obtained in this way, 3.33 kg of hexamethyldisiloxane was then dropped while stirring. Subsequently, the mixture obtained in this way was poured into a hobbock and tightly closed with a screw cap with a seal.

The hobbock was stored at 30° C. for 21 days.

After this period, the stored mixture was filled back into the reactor. The pressure was then gradually reduced to 280 mbar, and the reaction mixture was heated to a temperature of 44° C. The C₁-C₆ alcohols (ethanol and especially methanol) present in the reaction mixture were distilled off. Simultaneously with distilling off the alcohol mixture, 1000 ml of ethanol was slowly added to the reaction mixture. The distilled alcohols were collected in a chilled receiver. Distillation was continued until no more alcohols condensed in the receiver under the selected reaction conditions. The reaction mixture was then allowed to cool to room temperature. It was stirred for 10 minutes. In each case, 100 ml of the silane blend was filled into a bottle with a capacity of 100 ml and screw cap closure with seal. After filling, the bottles were tightly closed.

### 2. Coloring

The following colorant was provided (preparation (B)).

### Preparation (B)

| | |
|---|---|
| Colorona Bordeaux, Merck, MICA, CI 77491 (IRON | 3.5 g |
| OXIDES) | |
| Hydroxyethyl cellulose (Natrosol 250 HR) | 1.0 g |
| Eco Smooth Satin (Ethylene/Sodium Acrylate Copolymer | 40.0 |
| 25% solution, Dow Chemical Company) | |
| PEG-12 Dimethicone (Xiameter OFX-0193) | 2.0 g |
| Water | Ad 100 g |

From each of the previously filled bottles with silane blend, 20 g were weighed out (preparation A). The ready-to-use stain was prepared by shaking 20 g of preparation (A) and 100 g of preparation (B), respectively (shaking for 3 minutes). This mixture was then left to stand for 5 minutes.

For the application, one strand of hair (Kerling dark brown) was dipped into the ready-to-use dye and left in it for 1 minute. After that, superfluous agent was stripped from each strand of hair. Then each strand of hair was washed with water and dried. Subsequently, the strands were visually evaluated under a daylight lamp. The following results were obtained:

Silane blend (SB) 10 g
Colorant (B) 100 g
Coloration: uniform burgundy red
Color intensity: high
Hiding power: medium

1. A process for the preparation of an agent for the treatment of keratinous material, comprising the following steps:
(1) mixing one or more organic C₁-C₆ alkoxy silanes with water to give a mixture,
(2) optionally, adding one or more cosmetic ingredients to the mixture,
(3) storing the mixture for a period of at least about 5 days to give a stored mixture,
(4) optionally, adding one or more cosmetic ingredients to the stored mixture; and
(5) partially or completely removing from the stored mixture one or more C₁-C₆ alcohols released by a reaction of the one or more organic C₁-C₆ alkoxy silanes and water in steps (1) to (4).

2. The process according to claim 1, wherein in step (1) one or more organic C₁-C₆ alkoxy silanes of formula (I) and/or (II) are mixed with water,
R₁R₂N-L-Si(OR₃)ₐ(R₄)_{b} (I)
where
R₁, R₂ independently represent a hydrogen atom or a C₁-C₆ alkyl group,
L is a linear or branched divalent C₁-C₂₀ alkylene group,
R₃, R₄ independently of one another represent a C₁-C₆ alkyl group,
a represents an integer from 1 to 3, and
b represents the difference of 3-a, and
(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),
where
each R₅, R₅′, R₆, and R₆′ independently represent a C₁-C₆ alkyl group,
each A A′, A″, and A′″ independently represent a linear or branched divalent C₁-C₂₀ alkylene group,
R₇ and R₈ each independently represent a hydrogen atom, a C₁-C₆ alkyl group, a hydroxy C₁-C₆ alkyl group, a C₂-C₆ alkenyl group, an amino C₁-C₆ alkyl group or a group of formula (III),
-(A″″)-Si(R₆″)_{d″}(OR₅″)_{c}″ (III),
where each R₅″ and R₆″ independently represents a C₁-C₆ alkyl group, A″″ independently represents a linear or branched divalent C₁-C₂₀ alkylene group, c″ stands for an integer from 1 to 3, and d″ represents the difference of 3-a,
c represents an integer from 1 to 3,
d represents the difference of 3-c,
c′ represents an integer from 1 to 3,
d′ represents the difference of 3-c′, and
e, f, g, and h each independently stand for 0 or 1,
provided that at least one of e, f, g, and h is different from 0.

3. The process of claim 1, wherein in step (1) one or more organic C₁-C₆ alkoxy silanes of formula (IV) are mixed with water,
R₉Si(OR₁₀)ₖ(R₁₁)ₘ (IV),
where
R₉ represents a C₁-C₁₂ alkyl group,
each R₁₀ represents a C₁-C₆ alkyl group,
each R₁₁ represents a C₁-C₆ alkyl group,
k represents an integer from 1 to 3, and
m represents the difference of 3-k.

4. The process according to claim 1, wherein in step (1) the one or more organic C₁-C₆ alkoxy silanes are mixed with water in a reaction vessel or reactor comprising a double-wall reactor, a reactor with external heat exchanger, a tubular reactor, a reactor with thin-film evaporator, a reactor with falling-film evaporator and/or a reactor with attached condenser.

5. The process according to claim 1, wherein in step (1) the one or more organic C₁-C₆ alkoxy silanes are mixed with from 0.10 to 0.80 molar equivalents of water (S-W), where the molar equivalents of water (S-W) is calculated according to the formula
$$S \text{-} W = \frac{mol ( Water )}{mol ( Silane ) \times n ( Alkoxy )}$$
where
mol(water) represents the molar quantity of water used in step (1)
mol(silanes) represents the total molar amount of C₁-C₆ alkoxy silanes used in in step (1), and
n(alkoxy) represents the stoichiometric=number of C₁-C₆ alkoxy groups of the one or more organic C₁-C₆ alkoxy silane.

6. The process according to claim 1, wherein in step (1) the one or more organic C₁-C₆ alkoxy silanes are mixed with water at a temperature of from about 20° C. to about 70° C.

7. The process according to claim 1 comprising step (2) of adding the one or more cosmetic ingredients to the mixture, wherein
one or more of the cosmetic ingredients is selected from the group of solvents, polymers, surface-active compounds, colorants, lipid components, pH regulators, perfumes, preservatives, plant extracts, and protein hydrolysates.

8. The process according to claim 7, wherein step (2) comprises adding to the mixture one or more solvents selected from the group of hexamethyldisiloxane, octamethyltrisiloxane, decamethyltetrasiloxane, hexamethylcyclotrisiloxane, octamethylcyclotetrasiloxane, and decamethylcyclopentasiloxane.

9. The process according to claim 1, wherein step (3) is further defined as storing the mixture for a period of at least about 8 days.

10. The process according to claim 9, wherein the mixture is stored at a temperature in the range of from about 15° C. to about 45° C.

11. The process according to claim 1 comprising step (4) of adding the one or more cosmetic ingredients to the stored mixture, wherein
one or more of the cosmetic ingredients is selected from the group of ethanol, isopropanol, and methanol.

12. The process according to claim 1, wherein in step (5) the partial or complete removal of the one or more C₁-C₆ alcohols is carried out at a temperature of from about 20° C. to about 70° C.

13. The process according to claim 1, wherein in step (5) the partial or complete removal of the one or more C₁-C₆ alcohols is carried out via distillation at a pressure of from about 10 to about 900 mbar.

14. The process according to claim 1, wherein steps (1)(5) prepares a preparation, and wherein the process further comprises a step of
(6) filling the preparation into a packaging unit selected from the group of a bottles, tubes, jars, cans, sachets, aerosol pressure containers, non-aerosol pressure containers, canisters, and hobbocks.

15. The process according to claim 1, comprising the steps of;
(1) the one or more organic C₁-C₆ alkoxy silanes with water to give the mixture,
(2) optionally, adding the one or more cosmetic ingredients to the mixture obtained in step (1),
(3) storing the mixture for a period of at least about 5 days to give the stored mixture,
(4) adding ethanol to the stored mixture, and
(5) partially or completely removing the one or more C₁-C₆ alcohols from the mixture,
wherein first step (1) is carried out, then optionally step (2) is carried out, then step (3) is carried out, and subsequently steps (4) and (5) are carried out simultaneously.

16. The process according to claim 1, wherein steps (1)-(5) prepares a preparation, and wherein the preparation is further defined as an agent for coloring keratinous material, maintaining keratinous material, or changing the shape of keratinous material.

17. A multicomponent packaging unit (kit-of-parts) for dyeing keratinous material, comprising, separately:
a first packaging unit comprising a cosmetic preparation (A) and
a second packaging unit comprising a cosmetic preparation (B),
where
the cosmetic preparation (A) of the first packaging unit is prepared according to the process of claim 1, and
the cosmetic formulation (B) comprises at least one colorant compound selected from the group of pigments, direct dyes, and/or oxidation dye precursors.

18. The process according to claim 1, wherein: (i) in step (1) the one or more organic C₁-C₆ alkoxy silanes are mixed with water at a temperature of from about 20 to about 65° C.: (ii) step (3) comprises storing the mixture for a period of at least about 8 days and at a temperature of from about 15 to about 45° C.: (iii) step (5) is carried out at a temperature of from about 20° C. to about 60° C. and a pressure of from about 10 to about 900 mbar: or (iv) any of (i)-(iii).

19. The process according to claim 1, wherein: (i) in step (1) the one or more organic C₁-C₆ alkoxy silanes are mixed with water at a temperature of from about 20 to about 45° C.: (ii) step (3) comprises storing the mixture for a period of at least about 8 days and at a temperature of from about 20 to about 45° C.: (iii) step (5) is carried out via distillation at a temperature of from about 20 to about 45° C. and a pressure of from about 10 to about 300 mbar: or (iv) any of (i)-(iii).

20. The process according to claim 19, comprising step (4), wherein step (4) comprises adding ethanol, isopropanol, and/or methanol to the stored reaction mixture prior to and/or during the distillation of step (5).
